(19) United States
(12) Patent Application Publication
(10) Pub. No.: US 2022/0362766 A1
(43) Pub. Date: Nov. 17, 2022

(54) Microfluidic Sensor for Continuous or Semi-Continuous Monitoring of Quality of an Aqueous Solution

(71) Applicant: University of South Australia, Adelaide (AU)

(72) Inventors: Craig Ian Priest, Athelstone (AU); Sait Elmas, Largs North (AU); Aneta Pospisilova, Mikulov (CZ); Aneta Anna Sekulska, Maciejowice (PL)

(21) Appl. No.: 17/771,390

(22) PCT Filed: Oct. 23, 2020
(86) PCT No.: PCT/AU2020/000126

§ 371 Date: Apr. 22, 2022

(30) Foreign Application Priority Data
Oct. 25, 2019 (AU) 2019904034

(51) Int. Cl. B01L 3/00; F04B 19/00; G01N 21/80; G01N 33/18

(52) U.S. Cl. CPC B01L 3/502715; F04B 19/006; G01N 21/80; G01N 33/182; B01L 2200/148; B01L 2200/16; B01L 2300/0867; B01L 2300/0883; G01N 2021/0346

(57) ABSTRACT

The present disclosure relates to a microfluidic device for measuring one or more parameters in a fluid sample, which includes a sample microfluidic channel disposed on a solid substrate, a reagent microfluidic channel disposed on a solid substrate, a mixing microfluidic channel disposed on a solid substrate, and an optical reading window located downstream of the mixing microfluidic channel, through which a response indicative of the parameter(s) change can be measured optically. The present disclosure also relates to an apparatus for measuring one or more parameters in a fluid sample which includes the microfluidic device as well as a method for measuring one or more parameters in a fluid sample through the device or the apparatus.

### CROSS-REFERENCE TO RELATED APPLICATIONS

This application is the United States national phase of International Application No. PCT/AU2020/000126 filed Oct. 23, 2020, and claims priority to Australian Provisional Patent Application No. 2019904034 filed Oct. 25, 2019, the disclosures of which are hereby incorporated by reference in their entirety.

### BACKGROUND OF THE INVENTION

### Field of the Invention

The present disclosure generally relates to devices, apparatus and methods for monitoring a quality of a fluid sample. In particular, the present disclosure relates to devices, apparatus and methods for continuous or semi-continuous monitoring of chemical and/or physical parameters in an aqueous solution. More particularly, the present disclosure relates to devices, apparatus and methods for continuous or semi-continuous photometric measurement of active chlorine, total chlorine and/or pH in an aqueous solution.

### Description of Related Art

In various endeavours, there is a need to monitor fluid quality, for instance, to measure a chemical parameter or a physical parameter in water. Chlorination is important for disinfection of recreation and drinking water and is strictly regulated.^{1,2} Furthermore, it must be carefully monitored to avoid outbreaks of waterborne disease.^{3,4}

The amount of organic and inorganic matter in a water body that can react with an amount of chlorine is called the “chlorine demand” Upon chlorine addition, after the chlorine demand is satisfied, the excess chlorine is referred to as “residual chlorine”. In an ever-changing water environment, like a swimming pool, the residual chlorine is available to react with germs, sweat, oils, urine, etc. and it is often called “available chlorine”. For a swimming pool to be ready for use the operator should maintain a delicate “water balance”—the chlorine amount, total alkalinity, calcium hardness, pH and total dissolved solids must be within specified lower and upper limits¹.

Active chlorine can be produced upon aqueous dissolution of inorganic hypochlorite salts, photocatalytically on Ag@AgCl and TiO₂ photoelectrodes,^{5-7,27-28} or directly via generation of molecular chlorine (Cl₂) at an electrode-water interface.²⁹⁻³¹ Chlorine gas that is generated at an anode surface immediately disproportionates in swimming pool water to form HOCl and HCl, acidifying the water. Active chlorine is photolytically cleaved upon exposure to ultraviolet radiation, i.e. sunlight, and consumed in redox reactions with other components of pool water, such as organic matter or nitrogen compounds. Active chlorine can also react with nitrogen-containing organics (urea, sweat, microorganisms) to form chloramines, termed ‘bound’ chlorine. Chloramine is also a disinfection agent but remains in solution longer than HOCl and has an inferior disinfection power.

Aqueous chlorine exists primarily as hypochlorous acid (HOCl) and hypochlorite ion (OCl⁻) and the relative ratio of hypochlorite ion to hypochlorous acid is related to the pH of the solution. The additive concentration of the two moieties is referred to as “active chlorine”. Organic nitrogen-containing species like amines, amino-acids, proteins, urea, introduced into a water body through sweat, urine, hair, etc. react with active chlorine to form organic/inorganic chloramines. The total chloramine concentration is called “combined chlorine”. The sum of “combined chlorine” and “active chlorine” concentration is called “total chlorine”. The “active”, “combined” and “total chlorine” concentrations are measured as “mg/L (ppm) as Cl_{2(aq.)}”, comparing the relative oxidizing capacity of the water relative to a solution of pure Cl_{2(aq.)}.⁶ The germicide power of “combined chlorine” is much lower than “active chlorine”. Additionally, “combined chlorine” has an objectionable taste and odour and can cause irritation of the eyes of swimmers.^{5,6} Thus, when the “combined chlorine” concentration is over an upper limit or the “active chlorine” concentration is under a lower limit a pool operator needs to take corrective action.

On the other hand, the disinfection power⁷ of active and combined chlorine species and their stability⁸⁻⁹ in water strongly depend on the pH. For swimming pools, the pH may vary over time, for example due to reduction of active chlorine upon exposure to ultraviolet radiation and redox reactions of active chlorine with other components of pool water, such as organic matter or nitrogen compounds. In addition, swimming pools must be buffered, usually with sodium bicarbonate. Therefore, active chlorine, total chlorine, and/or pH must be monitored regularly.¹²⁻¹⁴

Furthermore, alkalinity and pH are two important factors in determining the suitability of water for irrigating plants. The generally accepted pH for irrigation water is between 5.5 and 7.5.¹⁰ Alkalinity is a measure of the water's ability to neutralize acidity. An alkalinity test measures the level of bicarbonates, carbonates, and hydroxides in water from the geologic materials of the aquifer from which the water is drawn, such as limestone and dolomite.¹¹

Numerous methods for the measurement of the residual chlorine have been developed. Amperiometric methods are considered the benchmark against which aqueous chlorine samples are tested.⁶ Amperiometric methods can be used for the continuous determination of active chlorine. However, amperiometric methods have a relatively higher cost and require greater operator skill; additionally, there is a greater interference from pH, T, p and chlorine concentration. These methods cannot accommodate abrupt changes in chlorine concentration and are not suitable for field use.¹⁵ A similar method is the non-selective electrode ORP (oxidation reduction potential) method.⁶ However, the ORP method cannot differentiate between active chlorine and combined chlorine and therefore the correlation between disinfection efficiency and ORP is poor. Additionally, the electrodes used in the ORP method are subject to drift, fouling and poisoning.⁶

Methods for chlorine measurement that are more suitable for home or in-field use are based on measuring changes in the concentration of a dye after exposure of the dye to a chlorine containing solution. Diethyl-1,4-phenylenediamine (DPD) is a dye that is commonly used for chlorine detection in swimming pools. There are some shortcomings with the use of DPD for continuous chlorine monitoring, namely the reagent is unstable and needs to be buffered, only a narrow range of chlorine concentration (i.e. 0-5 ppm) can be measured, and chloramines interfere with the measurements.

In general, prior art active chlorine sensors needed to be paired with some form of pH compensation because active chlorine exists as hypochlorite ion and hypochlorous acid with the relative ratio of the two being dependent upon the pH of the solution. Below pH 6, active chlorine is effectively 100% hypochlorous acid, while above pH 10, active chlorine is effectively 100% hypochlorite ion. The requirement that pH must be known to some degree complicates chlorine measurement and increases the expense of measurement systems.

Effective swimming pool management and safety demands a continuous and automated water quality sensor that can measure (at least) active chlorine, total chlorine, and pH. Most existing technologies are expensive, require maintenance, and many domestic pool owners still rely on test-strips and other manual low-cost sensors. Progress in water monitoring is being made by groups using photometric sensors for reliable chemical analysis of environmental water.^{16, 17} Functional materials (e.g. quantum dots) have also been used in the photometric method to enhance sensitivity to chlorine.^{18, 19}

US2013/0330245 A1 discloses a chip-based water analysis device including a fluid channel disposed therein, which can be used to determine chlorine content of water via reaction with an indicator dye, such as diethyl-1,4-phenylenediamine (DPD) and optical testing of light absorption of the coloured product. This device employs pre-loaded reagents such as an indicator, a buffer and a quenching agent within its fluid channel, which requires accurately and precisely determining the position of the fluid sample within the device. Furthermore, this device is a single use device and, as such, it is not configured for online continuous or semi-continuous measurement and the potential for consumption of the reagent(s) associated with such use. Further, the shortcomings outlined above make the use of DPD not suitable for continuous chlorine monitoring.

Therefore, the above challenges highlight a need for microfluidic sensors for continuous or semi-continuous monitoring quality of an aqueous solution. Alternatively, or in addition, there is a need for microfluidic sensors that can be used for online continuous or semi-continuous measurement of active chlorine, total chlorine, and/or pH in water. Alternatively, or in addition, there is a need for microfluidic sensors that require relatively small consumption of sample and reagents for measurement, for example microliter sample and reagent volumes per measurement.

### SUMMARY OF THE INVENTION

In a first aspect, provided herein is a microfluidic device for measuring pH in a fluid sample, the device comprising:

a sample microfluidic channel disposed on a solid substrate and configured to transfer the fluid sample to be analysed,

a pH indicator microfluidic channel disposed on a solid substrate and configured to transfer a pH indicator solution capable of responding to pH in the fluid sample to produce a pH measurement solution having a response indicative of the pH of the fluid sample,

a mixing microfluidic channel disposed on a solid substrate and in fluid communication with the sample microfluidic channel and the pH indicator microfluidic channel, the mixing microfluidic channel being configured to mix the fluid sample to be analysed with the pH indicator solution under conditions suitable for the pH indicator to respond to pH in the fluid sample to produce a pH measurement solution having a response indicative of the pH, and

an optical reading window in fluid communication with an outlet of the mixing microfluidic channel, through which the response indicative of the pH change can be measured optically.

In certain embodiments of the first aspect, the device is configured to minimise backflow of the fluid sample and the pH indicator solution therein. Backflow of the fluid sample and the pH indicator solution in the device can be minimised by forming a pressure gradient from a higher pressure inlet end of the microfluidic channels to a lower pressure outlet end of the microfluidic channels. For example, a pressure gradient may be formed in the microfluidic channels using high-precision pumping and valving. For example, when all feeding pumps are stopped a pressure gradient will be formed within the device and this minimises backflow of the fluid sample and the pH indicator solution therein.

In another example, one or more of the sample microfluidic channel, the pH indicator microfluidic channel and the mixing microfluidic channel may have a relatively high flow resistance. In certain embodiments, the mixing microfluidic channel is serpentine in form, and this provides a high flow resistance which helps prevent backflow of the fluid sample and the pH indicator solution therein.

In certain embodiments of the first aspect, the device further comprises one or more high volume reagent storage channel(s) in fluid connection with the pH indicator microfluidic channel. The reagent storage channel(s) are configured to store a volume of the pH indicator solution.

In certain embodiments of the first aspect, the microfluidic device comprises a sample high flow resistance microfluidic channel disposed on a solid substrate and configured to transfer the fluid sample to be analysed, and a pH indicator high flow resistance microfluidic channel disposed on a solid substrate and configured to transfer a pH indicator solution capable of responding to pH in the fluid sample to produce a pH measurement solution having a response indicative of the pH, the mixing high flow resistance microfluidic channel is in fluid communication with the sample high flow resistance microfluidic channel and the pH indicator high flow resistance microfluidic channel.

In certain embodiments of the first aspect, the sample microfluidic channel, the pH indicator microfluidic channel and the mixing microfluidic channel are serpentine in form and each serpentine channel provides some flow resistance at an upstream end of the device to thereby minimise backflow of the fluid sample and the pH indicator solution therein.

In certain embodiments of the first aspect, the microfluidic device comprises a waste microfluidic channel located downstream of the optical reading window.

In certain embodiments of the first aspect, the flow resistance of the sample high flow resistance microfluidic channel, the pH indicator high flow resistance microfluidic channel, and the mixing high flow resistance microfluidic channel are sufficient to minimise backflow of the fluid sample and the pH indicator solution during operation.

In certain embodiments of the first aspect, the sample microfluidic channel, the pH indicator microfluidic channel, the mixing microfluidic channel, and if present, the waste microfluidic channel are configured to allow diffusive mixing.

In certain embodiments of the first aspect, the cross-section of the mixing microfluidic channel is of greater size than those of the sample microfluidic channel, the pH indicator microfluidic channel, and if present, the waste microfluidic channel.

In certain embodiments of the first aspect, the sample microfluidic channel, the pH indicator microfluidic channel, and the waste microfluidic channel have a cross-section of 103 μm×214 μm, and the mixing high flow resistance microfluidic channel has a cross-section of 117 μm×245 μm.

In certain embodiments of the first aspect, the mixing microfluidic channel is configured so that the residence time from mixing the fluid sample and the pH indicator solution to arriving at the optical window is longer than the minimum diffusive mixing time.

In certain embodiments of the first aspect, the solid substrate is made from a material selected from the group consisting of glass, quartz, metal (e.g. stainless steel), ceramic, silicon, and polymers. The polymers may be selected from thermoplastic polymers such as polystyrene, polycarbonate, polymethyl methacrylate and polyethylene glycol diacrylate, thermoset polymers such as polyester, elastomers such as polydimethylsiloxane (PDMS) and polyurethane, and cyclic olefin copolymers.

In certain embodiments of the first aspect, the microfluidic device comprises a measuring chamber comprising the optical reading window and configured to receive the pH measurement solution and through which the response indicative of pH in the pH measurement solution can be measured optically.

In certain embodiments of the first aspect, the microfluidic device is for online measuring pH in an aqueous solution sample.

In a second aspect, provided herein is an apparatus for measuring pH in a fluid sample, which comprises the microfluidic device of the first aspect.

In certain embodiments of the second aspect, the apparatus comprises a light source and a detector. For example, the light source is a LED and the detector is a photodiode.

In certain embodiments of the second aspect, the apparatus comprises one or more pumping means to pump the fluid sample and the pH indicator solution through the device. If desired, the pumping means can be selected from a peristaltic pump, a syringe pump and a micro-syringe pump. Alternatively, the pumping means may comprise methods for moving liquids known in the art, such as capillarity, wetting (including electrowetting), and transpiration (i.e. controlled evaporation).

In a third aspect, provided herein is a method of measuring pH in a fluid sample, which includes using the microfluidic device of the first aspect or the apparatus of the second aspect. The method is further adaptable to online continuous or semi-continuous measurement of pH with economic reagent consumption. The fluid sample described herein can be an aqueous solution sample, for instance a sample taken from a swimming pool or an irrigation water sample. In some cases, measurement of a pH in the range between 6 and 8.5 may be of interest. In some cases, measurement of a pH in the range between 5.5 and 7.5 may be of interest.

In certain embodiments of the third aspect, the response indicative of the pH is a colour change.

In certain embodiments of the third aspect, the pH indicator is selected from the group consisting of thymol blue, methyl yellow, phenol red, congo red, methyl orange, methyl red, neutral red and alizarine yellow R. For example, when the pH indicator is phenol red, the pH indicator solution can have a pH of 6.4 and can have a concentration of 41.2 mg/L phenol red. The concentration here is not important. When the pH indicator is phenol red, the absorbance of peaks centred at 432 nm, 560 nm and 650 nm are measured, logarithm of relative peak intensity ln((A₄₃₂−A₆₅₀)/(A₅₆₀−A₆₅₀)) can be calculated, and the average value used to calculate the pH.

In certain embodiments of the third aspect, the mixing ratio of the fluid sample and the pH indicator solution is 1:1.

In a fourth aspect, provided herein is a microfluidic device for measuring more than one parameter in a fluid sample, the device comprising:

a sample microfluidic channel disposed on a solid substrate and configured to transfer the fluid sample to be analysed,

a first reagent microfluidic channel disposed on a solid substrate and configured to transfer a first reagent solution capable of reacting with a chemical substance in the fluid sample to produce a first parameter measurement solution having a response that is indicative of the first parameter in the fluid sample,

a second reagent microfluidic channel disposed on a solid substrate and configured to transfer a second reagent solution capable of reacting with a chemical substance in the fluid sample to produce a second parameter measurement solution having a response that is indicative of the second parameter in the fluid sample,

a mixing microfluidic channel disposed on a solid substrate and in fluid communication respectively with the sample microfluidic channel, the first reagent microfluidic channel and the second reagent microfluidic channel, which is configured to mix the fluid sample separately with the first reagent solution and the second reagent solution under conditions suitable for some of the first reagent to react with a chemical substance in the fluid sample to produce a first parameter measurement solution having a response that is indicative of the first parameter in the fluid sample and suitable for some of the second reagent to react with a chemical substance in the fluid sample to produce a second parameter measurement solution having a response that is indicative of the second parameter in the fluid sample, and

an optical reading window in fluid communication with an outlet of the mixing microfluidic channel, through which the first parameter and the second parameter can be optically measured.

The chemical parameters described herein may be those capable of being optically measured, which include, but are not limited to, chlorine concentration, pH and alkalinity. The microfluidic device then can be configured to measure two, three or more parameters as desired.

In certain embodiments of the fourth aspect, the microfluidic device comprises a waste microfluidic channel located downstream of the optical reading window.

In certain embodiments of the fourth aspect, the microfluidic device comprises a third reagent microfluidic channel disposed on the solid substrate and configured to transfer a third reagent solution capable of reacting with a chemical substance in the fluid sample to produce a third parameter measurement solution having a response that is indicative of the third parameter in the fluid sample, and the mixing microfluidic channel is also in fluid communication with the third reagent solution and is configured to mix the fluid sample with the third reagent solution suitable for some of the third reagent to react with a chemical substance in the fluid sample to produce a third parameter measurement solution having a response that is indicative of the third parameter in the fluid sample.

In certain embodiments of the fourth aspect, the sample microfluidic channel, the first reagent microfluidic channel, the second reagent microfluidic channel and, if present, the third reagent microfluidic channel are disposed on the same solid substrate.

In certain embodiments of the fourth aspect, the mixing microfluidic channel and the optical reading window are disposed on a solid substrate different from the solid substrate(s) upon which the sample microfluidic channel, the first reagent microfluidic channel, the second reagent microfluidic channel and, if present, the third reagent microfluidic channel are disposed.

In certain embodiments of the fourth aspect, the sample microfluidic channel, the first reagent microfluidic channel, the second reagent microfluidic channel, the third reagent microfluidic channel (if present), the mixing microfluidic channel and the optical reading window are disposed on the same solid substrate.

In certain embodiments of the fourth aspect, the device is configured to minimise backflow of the fluid sample, the first reagent, the second reagent and, if present, the third reagent therein. Backflow of the fluid sample, the first reagent, the second reagent and, if present, the third reagent in the device can be minimised by forming a pressure gradient from a higher pressure inlet end of the microfluidic channels to a lower pressure outlet end of the microfluidic channels. For example, a pressure gradient may be formed in the microfluidic channels using high-precision pumping and valving. For example, when all feeding pumps are stopped a pressure gradient will be formed within the device and this minimises backflow of the fluid sample, the first reagent, the second reagent and, if present, the third reagent therein.

In another example, one or more of the sample microfluidic channel, the first reagent microfluidic channel, the second reagent microfluidic channel, the third reagent microfluidic channel (if present), and the mixing microfluidic channel, may have a relatively high flow resistance. In certain embodiments, the mixing microfluidic channel is serpentine in form, and this provides a high flow resistance which helps prevent backflow of the fluid sample, the first reagent, the second reagent and, if present, the third reagent therein.

In certain embodiments of the fourth aspect, the device further comprises one or more high volume reagent storage channels in fluid connection with any one or more of each of the first reagent microfluidic channel, the second reagent microfluidic channel, and the third reagent microfluidic channel (if present), the reagent storage channel(s) configured to store a volume of the first reagent microfluidic channel, the second reagent microfluidic channel or the third reagent microfluidic channel (if present), respectively.

In certain embodiments of the fourth aspect, the sample microfluidic channel, the first reagent microfluidic channel, the second reagent microfluidic channel, the third reagent microfluidic channel (if present), and the mixing microfluidic channel are serpentine in form.

In certain embodiments of the fourth aspect, the sample microfluidic channel, the first reagent microfluidic channel, the second reagent microfluidic channel, the third reagent microfluidic channel (if present), and the mixing microfluidic channel are configured to allow diffusive mixing.

In certain embodiments of the fourth aspect, the cross-section of the mixing microfluidic channel is of greater size than those of the sample microfluidic channel, the first reagent microfluidic channel, the second reagent microfluidic channel, the third reagent microfluidic channel (if present), and the waste microfluidic channel.

In certain embodiments of the fourth aspect, the mixing microfluidic channel is configured so that the residence time from mixing the fluid sample separately with the first reagent solution, the second reagent solution and, if present, the third reagent solution to arriving at the optical reading window is longer than the characteristic time for diffusive mixing. This is believed to be advantageous for delivering a homogeneous stream to the optical reading window.

In a fifth aspect, provided herein is an apparatus for measuring more than one parameter in a fluid sample, which comprises the microfluidic device of the fourth aspect.

In certain embodiments of the fifth aspect, the apparatus comprises a light source and a detector. For example, the light source is a LED and the detector is a photodiode.

In certain embodiments of the fifth aspect, the apparatus comprises one or more pumping means to pump the fluid sample, the first reagent solution, the second reagent solution and, if present, the third reagent solution through the device. If desired, the pumping means can be selected from a peristaltic pump, syringe pump and micro-syringe pump. Alternatively, the pumping means may comprise methods for moving liquids known in the art, such as capillarity, wetting (including electrowetting), and transpiration (i.e. controlled evaporation).

In a sixth aspect, provided herein is a microfluidic device for measuring an amount of active chlorine, an amount of total chlorine and pH in a fluid sample, the device comprising:

a sample microfluidic channel disposed on a solid substrate and configured to transfer the fluid sample to be analysed,

a first reagent microfluidic channel disposed on a solid substrate and configured to transfer a first indicator dye solution capable of reacting with any active chlorine in the fluid sample to produce an active chlorine measurement solution having a reduced indicator dye concentration that is indicative of the amount of active chlorine in the fluid sample,

a second reagent microfluidic channel disposed on a solid substrate and configured to transfer a second indicator dye solution capable of reacting with any total chlorine in the fluid sample to produce a total chlorine measurement solution having a reduced indicator dye concentration that is indicative of the amount of total chlorine in the fluid sample,

a third reagent microfluidic channel disposed on a solid substrate and configured to transfer a pH indicator solution capable of responding to pH in the fluid sample to produce a pH measurement solution having a response indicative of the pH,

a mixing microfluidic channel disposed on a solid substrate and in fluid communication respectively with the sample microfluidic channel, the first reagent microfluidic channel, the second reagent microfluidic channel and the third reagent microfluidic channel, which is configured to mix the fluid sample separately with the first indicator dye solution, the second indicator dye solution and the pH indicator solution under conditions suitable for some of the indicator dye in the first indicator dye solution to react with any active chlorine in the fluid sample to produce an active chlorine measurement solution having a reduced indicator dye concentration that is indicative of the amount of active chlorine in the fluid sample, suitable for some of the indicator dye in the second indicator dye solution to react with any total chlorine in the fluid sample to produce a total chlorine measurement solution having a reduced indicator dye concentration that is indicative of the amount of total chlorine in the fluid sample, and suitable for the pH indicator to respond to pH in the fluid sample to produce a pH measurement solution having a response indicative of the pH, and

an optical reading window in fluid communication with an outlet of the mixing microfluidic channel, through which the amount of active chlorine, the amount of total chlorine and pH in the fluid sample can be optically measured.

In certain embodiments of the sixth aspect, the sample microfluidic channel, the first reagent microfluidic channel, the second reagent microfluidic channel and the third reagent microfluidic channel are disposed on the same solid substrate.

In certain embodiments of the sixth aspect, the mixing microfluidic channel and the optical reading window are disposed on a solid substrate different from the solid substrate(s) upon which the sample microfluidic channel, the first reagent microfluidic channel, the second reagent microfluidic channel and the third reagent microfluidic channel are disposed.

In certain embodiments of the sixth aspect, the sample microfluidic channel, the first reagent microfluidic channel, the second reagent microfluidic channel and the third reagent microfluidic channel, the mixing microfluidic channel and the optical reading window are disposed on the same solid substrate.

In certain embodiments of the sixth aspect, the microfluidic device is a multilayer microfluidic device comprising first and second outer chips and first and second intermediate chips and wherein the sample microfluidic channel, the first reagent microfluidic channel, the second reagent microfluidic channel, and the third reagent microfluidic channel are disposed on the first intermediate chip, and the mixing microfluidic channel and the optical reading window are disposed on the second intermediate chip.

In certain embodiments of the sixth aspect, the microfluidic device comprises a waste microfluidic channel located downstream of the optical reading window.

In certain embodiments of the sixth aspect, the device is configured to minimise backflow of the fluid sample, the first reagent, the second reagent, and the third reagent therein. Backflow of the fluid sample, the first reagent, the second reagent, and the third reagent in the device can be minimised by forming a pressure gradient from a higher pressure inlet end of the microfluidic channels to a lower pressure outlet end of the microfluidic channels. For example, a pressure gradient may be formed in the microfluidic channels using high-precision pumping and valving. For example, when all feeding pumps are stopped a pressure gradient will be formed within the device and this minimises backflow of the fluid sample, the first reagent, the second reagent, and the third reagent therein.

In another example, one or more of the sample microfluidic channel, the first reagent microfluidic channel, the second reagent microfluidic channel, the third reagent microfluidic channel, and the mixing microfluidic channel may have a relatively high flow resistance. In certain embodiments, the mixing microfluidic channel is serpentine in form, and this provides a high flow resistance which helps prevent backflow of the fluid sample, the first reagent, the second reagent, and the third reagent therein.

In certain embodiments of the sixth aspect, the device further comprises one or more high volume reagent storage channel in fluid connection with any one or more of each of the first reagent microfluidic channel, the second reagent microfluidic channel, and the third reagent microfluidic channel, the reagent storage channel(s) is configured to store a volume of the first reagent microfluidic channel, the second reagent microfluidic channel or the third reagent microfluidic channel, respectively.

In certain embodiments of the sixth aspect, the sample microfluidic channel, the first reagent microfluidic channel, the second reagent microfluidic channel, the third reagent microfluidic channel, and the mixing microfluidic channel are serpentine in form.

In certain embodiments of the sixth aspect, the sample microfluidic channel, the first reagent microfluidic channel, the second reagent microfluidic channel, the third reagent microfluidic channel, the mixing microfluidic channel and, if present, the waste microfluidic are configured to allow diffusive mixing.

In certain embodiments of the sixth aspect, the cross-section of the mixing microfluidic channel is of greater size than those of the sample microfluidic channel, the first reagent microfluidic channel, the second reagent microfluidic channel, the third reagent microfluidic channel and, if present, the waste microfluidic channel.

In certain embodiments of the sixth aspect, the sample microfluidic channel, the first reagent microfluidic channel, the second reagent microfluidic channel, the third reagent microfluidic channel and, if present, the waste microfluidic channel have a cross-section of 103 μm×214 μm, and the mixing microfluidic channel has a cross-section of 117 μm×245 μm.

In certain embodiments of the sixth aspect, the mixing microfluidic channel is configured so that the residence time from mixing the fluid sample separately with the first indicator dye solution, the second indicator dye solution and the pH indicator solution to arriving at the optical reading window is longer than the characteristic time for diffusive mixing.

In certain embodiments of the sixth aspect, the solid substrate is made from a material selected from the group consisting of glass, quartz, metal (e.g. stainless steel), ceramic, silicon, and polymers.

In certain embodiments of the sixth aspect, the microfluidic device is for online continuous or semi-continuous measuring of an amount of active chlorine, an amount of total chlorine and pH in an aqueous solution sample. In some cases, the sample is taken from a swimming pool.

In a seventh aspect, provided herein is an apparatus for measuring an amount of active chlorine, an amount of total chlorine and pH in a fluid sample, which comprises the microfluidic device of the sixth aspect.

In certain embodiments of the seventh aspect, the apparatus comprises a light source and a detector. For example, the light source is a LED and the detector is a photodiode.

In certain embodiments of the seventh aspect, the apparatus comprises one or more pumping means to pump the fluid sample, the first reagent solution, the second reagent solution and, if present, the third reagent solution through the device. If desired, the pumping means can be selected from a peristaltic pump, syringe pump and micro-syringe pump. Alternatively, the pumping means may comprise methods for moving liquids known in the art, such as capillarity, wetting (including electrowetting), and transpiration (i.e. controlled evaporation).

In an eighth aspect, provided herein is a method of measuring an amount of active chlorine, an amount of total chlorine and pH in a fluid sample, which includes using the microfluidic device of the sixth aspect or the apparatus of the seventh aspect. The method is further adaptable to online continuous or semi-continuous measuring of an amount of active chlorine, an amount of total chlorine and pH in a fluid sample with economic reagent consumption. The fluid sample described herein can be an aqueous solution sample, for instance a sample taken from a swimming pool.

In certain embodiments of the eighth aspect, each of the first indicator dye and the second indicator dye is selected from an organic azo dye, an organic amine dye, and a thioninium dye. For example, the organic azo dye can be methyl orange, the organic amine dye can be DPD, and the thioninium dye can be methylene blue.

In certain embodiments of the eighth aspect, the first indicator dye solution is an unbuffered methyl orange solution containing a bromide, such as sodium bromide and potassium bromide. For example, the first indicator dye solution can be an unbuffered 100 ppm methyl orange solution containing 1000 ppm sodium bromide.

In certain embodiments of the eighth aspect, the second indicator dye solution comprises a combined chlorine release agent. The combined chlorine release agent described herein may be any reagent that releases chemically bound chlorine or forms activated bound chlorine, such as bromochloramine. For example, the combined chlorine release agent comprises a solution containing bromide ions (Br−), such as a potassium bromide (KBr) solution.

In certain embodiments of the eighth aspect, the second indicator dye solution is a buffered acidified methyl orange solution containing a bromide. In some cases, the second indicator dye solution is a buffered acidified 100 ppm methyl orange solution containing 4000 ppm sodium bromide.

In certain embodiments of the eighth aspect, the response indicative of the pH is a colour change. In some cases, the pH to be measured is in the range between 6 and 8.5.

In certain embodiments of the eighth aspect, the pH indicator is selected from the group consisting of thymol blue, methyl yellow, phenol red, congo red, methyl orange, methyl red, neutral red and alizarine yellow R. For example, when the pH indicator is phenol red, the pH indicator solution can have a pH of 6.4 and can have a concentration of 41.2 mg/L phenol red. When the pH indicator is phenol red, the absorbance of peaks centred at 432 nm, 560 nm and 650 nm are preferably recorded, logarithm of relative peak intensity ln((A₄₃₂−A₆₅₀)/(A₅₆₀−A₆₅₀)) can be calculated, and the average value used to calculate the pH.

In certain embodiments of the eighth aspect, for measuring an amount of active chlorine and an amount of total chlorine, the 1:3 mixing ratio of the first indicator solution and the fluid sample is used for the fluid sample containing less than 8 ppm active chlorine, the 1:1 mixing ratio of the first indicator solution and the fluid sample is used for the fluid sample containing at least 8 ppm active chlorine.

In certain embodiments of the eighth aspect, for measuring pH, the 1:1 mixing ratio of the pH indicator solution and the fluid sample is used.

In certain embodiments of the eighth aspect, the detection wavelength for measuring an amount of active chlorine and/or an amount of total chlorine is set at the isosbestic point of methyl orange at 469 nm.

In certain embodiments of the eighth aspect, the absorbance at 650 nm as background was subtracted from the absorbance at 469 nm, and the average values are used for measuring an amount of active chlorine and an amount of total chlorine.

For a better understanding of the embodiments, together with other and further features and advantages thereof, reference is made to the following description, taken in conjunction with the accompanying drawings. The scope of the invention will be pointed out in the appended claims

### BRIEF DESCRIPTION OF THE DRAWINGS

Non-limiting embodiments of the present invention will be discussed with reference to the accompanying figures wherein:

FIG. 1 shows a schematic representation of a microfluidic device for measuring pH in a fluid sample.

FIG. 2 shows a photograph of the microfluidic device shown schematically in FIG. 1.

FIG. 3 is a representation of each layer of the thermally bonded, 5-layer borosilicate device shown in FIGS. 1 and 2 and shows the length of the mixing microfluidic channel and the dimensions of the reagent storage channel.

FIG. 4 shows a perspective view of a microfluidic device for sensing of active chlorine, total chlorine and pH and includes an enlargement of the section of the device containing the mixing microfluidic channel and the optical reading window.

FIG. 5 shows (a) a perspective view of a microfluidic device for sensing of active chlorine, total chlorine and pH, and (b) a photograph of the thermally bonded, 4-layer borosilicate device.

FIG. 6 is a representation of each layer of the thermally bonded, 4-layer borosilicate device shown in FIG. 2 and shows the length of the respective microfluidic channels.

FIG. 7 shows isobestic point of 469 nm for methyl orange. The spectra for pH 7 to 11 overlap and are represented by a single black line.

FIG. 8 shows phenol red absorbance spectra after mixing 1:1 with a pH adjusted swimming pool sample.

FIG. 9 shows active chlorine calibrations using sodium hypochlorite standards and 100 ppm methyl orange at neutral pH. Sample/methyl orange flow and volume ratios (S/MO) are indicated in the legend, with notation ‘off’ and ‘on’ referring to off-chip and on-chip experiments, respectively.

FIG. 10 shows total chlorine calibrations using sodium hypochlorite standards and 100 ppm methyl orange buffered at pH 4. Sample/methyl orange flow and volume ratios (S/MO) are indicated in the legend, with notation ‘off’ and ‘on’ referring to off-chip and on-chip experiments, respectively.

FIG. 11 shows calibration for on-chip and off-chip pH analysis using phenol red (A₁=432 nm, A₂=560 nm).

FIGS. 12A and B show comparisons of active chlorine and total chlorine measurements against DPD and iodometry results, respectively. The open and closed circles represent the off-chip and on-chip methods, respectively. The triangles represent the AC7® test strips.

FIG. 13 shows comparison of pH measurements against precision pH electrode results. The open and closed circles represent the off-chip and on-chip methods, respectively. The triangles represent the AC7® test strips.

### DESCRIPTION OF THE INVENTION

The disclosure arises from the inventors' research into the use of microfluidic devices to measure chlorine concentration and/or pH in aqueous samples including, but not limited to, swimming pool water, waste water, and municipal water supplies. Chlorine concentration and pH are only two examples of a chemical parameter that can be measured using the microfluidic devices, and other parameters, such as alkalinity and the concentration for another oxidant in solution can also be measured. In specific embodiments, the inventors have developed microfluidic devices that are accurate and reliable, cost effective and allow for continuous, real-time measurement or monitoring of active chlorine content, combined chlorine, and pH or real-time measurement or monitoring of active chlorine content, total chlorine content and pH with less reagent consumption. The following description will provide more details about the microfluidic devices, which are intended only by way of example.

As used herein, the term “microfluidic”, and variants thereof, means that the chip, device, apparatus, substrate or related apparatus contains fluid control features that have at least one dimension that is sub-millimetre and, typically less than 100 μm, and greater than 1 μm. Furthermore, the term “microchannel”, and variants thereof, means a channel having at least one dimension that is sub-millimetre and, typically less than 100 μm, and greater than 1 μm.

As used herein, the term “high flow resistance” means that the flow resistance within one or more channels is sufficient to minimise or even prevent backflow of a fluid within one or more of the microfluidic channel. This will be explained later in detail.

As used herein, the term “fluid” means that a sample that can flow through one or more of the microfluidic channels under the action of pressure drop. As a non-limiting example, the fluid can be an aqueous solution, such as municipal water and water from a swimming pool.

Microfluidic Devices and Apparatuses Comprising Thereof

1. Microfluidic Devices for Measuring pH in a Fluid Sample

Described herein is a microfluidic device for measuring pH in a fluid sample, the device comprising: a sample microfluidic channel disposed on a solid substrate and configured to transfer the fluid sample to be analysed; a pH indicator microfluidic channel disposed on a solid substrate and configured to transfer a pH indicator solution capable of responding to pH in the fluid sample to produce a pH measurement solution having a response indicative of the pH of the fluid sample; a mixing microfluidic channel disposed on a solid substrate and in fluid communication with the sample microfluidic channel and the pH indicator microfluidic channel, the mixing microfluidic channel being configured to mix the fluid sample to be analysed with the pH indicator solution under conditions suitable for the pH indicator to respond to pH in the fluid sample to produce a pH measurement solution having a response indicative of the pH, and an optical reading window in fluid communication with an outlet of the mixing microfluidic channel, through which the response indicative of the pH change can be measured optically.

If desired, the microfluidic device comprises a waste microfluidic channel located downstream of the optical reading window.

The microfluidic device may also comprise a measuring chamber comprising the optical reading window and configured to receive the pH measurement solution and through which the response indicative of pH in the pH measurement solution can be optically measured.

The solid substrate(s) used for the microfluidic device described herein can be made from glass, quartz, metal (e.g. stainless steel), ceramic, silicon, and polymers. The polymers may be selected from thermoplastic polymers such as polystyrene, polycarbonate, polymethyl methacrylate and polyethylene glycol diacrylate, thermoset polymers such as polyester, elastomers such as polydimethylsiloxane (PDMS) and polyurethane, and cyclic olefin copolymers.

The solid substrate(s) can be in the form of a chip. If there is more than one solid substrate, the two or more substrates may be connected to one another in series or parallel using suitable tubing and connectors, as is known in the art. For example, a through-hole can be used to connect an upper solid substrate and a lower solid substrate.

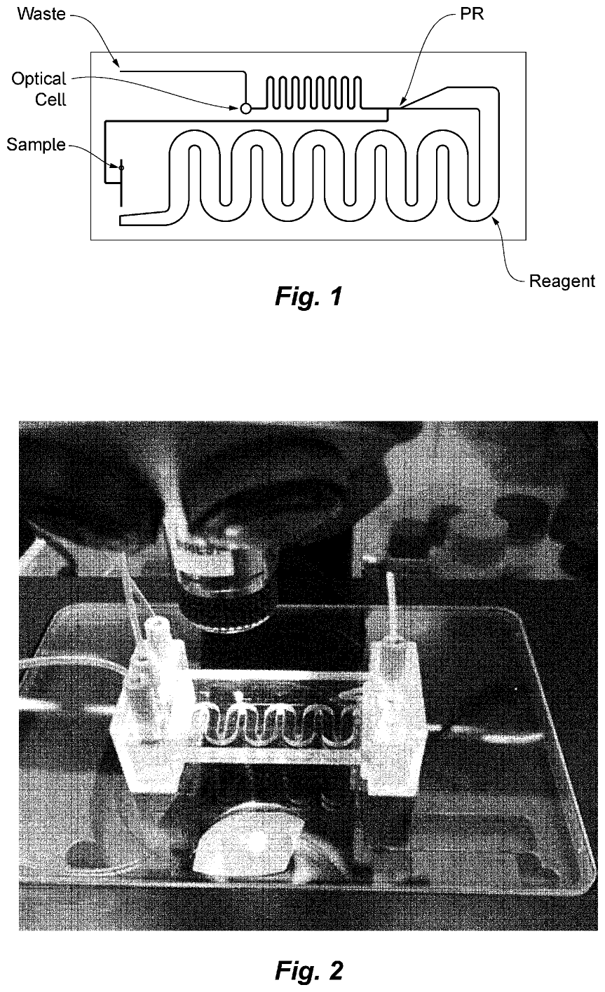

FIG. 1 shows one embodiment of a microfluidic device for measuring pH in a fluid sample. The microfluidic device comprises:
a sample microfluidic channel (designated as “sample”) disposed on a solid substrate and configured to transfer the fluid sample to be analysed,
a pH indicator microfluidic channel (designated as “PR”) disposed on a solid substrate and configured to transfer a pH indicator solution capable of responding to pH in the fluid sample to produce a pH measurement solution having a response indicative of the pH,
a mixing microfluidic channel (shown as a serpentine channel) disposed on a solid substrate and in fluid communication with the sample microfluidic channel and the pH indicator microfluidic channel, the microfluidic channel being configured to mix the fluid sample to be analysed with a pH indicator solution under conditions suitable for the pH indicator to respond to pH in the fluid sample to produce a pH measurement solution having a response indicative of the pH,
an optical reading window (designated as “optical cell”) in fluid communication with the mixing microfluidic channel, through which the response indicative of the pH change can be measured optically, and
a waste microfluidic channel (designated as “waste”) located downstream of the optical reading window,
   wherein the sample microfluidic channel, the pH indicator microfluidic channel, the mixing microfluidic channel, and the waste microfluidic channel are disposed on the same solid substrate, and the mixing microfluidic channel is serpentine in form.

In the illustrated embodiments, the microfluidic device further comprises a reagent storage channel (designated as “reagent”).

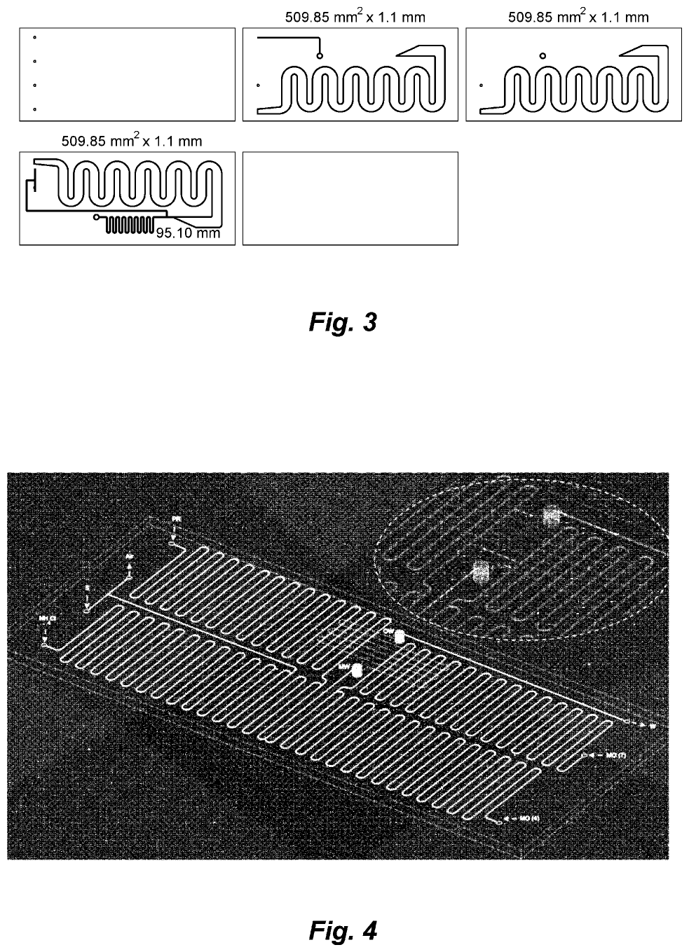

More specifically, the microfluidic device shown in FIG. 1 is a multilayer microfluidic device as shown in FIG. 3. The device comprises first and second outer chips and three intermediate chips. The sample microfluidic channel, the pH indicator microfluidic channel, the mixing microfluidic channel, part of the optical reading window and part of the reagent storage channel are disposed on a lower intermediate chip. Part of the reagent storage channel and part of the optical reading window are disposed on a middle intermediate chip. Part of the reagent storage channel, part of the optical reading window and the waste microfluidic channel are disposed on an upper intermediate chip.

For the embodiment shown in FIGS. 1 to 3, the mixing microfluidic channel has a length of about 95 mm. This configuration allows for complete diffusive mixing before measurement of the pH. The configuration will be discussed further under the section titled “Chip function”. The optical cell has a 2.2 mm path length. It is readily understood that longer path length will inevitably increase the sensitivity.

Backflow of the fluid sample and the pH indicator solution during operation can be minimised through flow resistance of the sample and/or the pH indicator in any of the microfluidic channels. For this purpose, the mixing microfluidic channel is serpentine in form for the embodiment shown in FIGS. 1 to 3.

For the purpose of precise measurement, a diffusive mixing within the mixing microfluidic channel is desirable, which facilitates delivering a homogeneous stream to the optical reading window. To achieve mixing prior to measurement of pH, the mixing microfluidic channel is configured so that the residence time from mixing the fluid sample and the pH indicator solution to arriving at the optical reading window is longer than the characteristic time for diffusive mixing. In the case of the embodiments shown in FIGS. 1 to 3, this can be achieved by making the length of the mixing microfluidic channel greater in size than those of the sample microfluidic channel, the pH indicator microfluidic channel, and if present, the waste microfluidic channel.

The microfluidic device described above may further comprise any one or more of:
a sample inlet port configured to receive the fluid sample to be analysed for pH,
a pH indicator inlet port configured to receive the pH indicator solution containing a pH indicator capable of responding to pH in the fluid sample to produce a pH measurement solution having a response indicative of the pH, and
a waste outlet port in fluid communication with the waste microfluidic channel and configured to allow the pH measurement solution to exit the device.

The fluid sample inlet port and the pH indicator solution inlet port can take any suitable form. For the embodiment shown in FIGS. 1 to 3, the fluid sample inlet port and the pH indicator solution inlet port can be formed in the first outer chip. In some embodiments, the inlet ports are in the form of apertures or openings in the first outer chip.

The present disclosure also relates to an apparatus comprising a microfluidic device described herein, which may further comprise any one or more of a pumping means, a light source and a detector. The sample and reagents may be transferred to the inlet ports and through the device under positive pressure provided by any suitable pump, by drawing the liquids through the device under vacuum, or by gravity feed. For example, the apparatus may comprise one or more pumping means to pump the fluid sample and the pH indicator solution through the device. A variety of pumping means suitable for this purpose are known in the art and, for example, can be selected from a peristaltic pump, a syringe pump and a micro-syringe pump. Examples include a syringe pump such as the ones from KD Scientific or a micro-syringe such as the ones under Gastight® from Hamilton Robotics. Alternatively, the pumping means may comprise methods for moving liquids known in the art, such as capillarity, wetting (including electrowetting), and transpiration (i.e. controlled evaporation).

A light source can be configured to project light through the optical reading window. The light source to be used is dependent upon the chromogenic assay at hand. For example, narrow-band emission LEDs of various wavelengths including red, blue and green may be used to illuminate chromophores having certain absorbance bands. Diode lasers may also be used as a source of electromagnetic radiation. Broad-band sources such as a Tungsten lamp may be coupled with filters to select wavelength used to probe a chromophore. Infra-red emitters may also be used. All of the foregoing may be used alone or in combination with each other, the choice dependent upon the assay/analyte to be detected. The detector can be employed to measure the absorbance of the solution reaching the optical reading window and may be a photodiode array spectrometer or a photodetector which is not wavelength selective. In the latter case, the incident light could be monochromatic. Examples for the detector include a custom-built micro-spectrophotometer based on an Olympus BH2-UMA frame and Ocean Optics Flame™ spectrophotometer.

The flow rates of the fluid sample and the pH indicator solution are independently controllable. The apparatus may further comprise at least one flow controller. The flow controller may include one or more valves, flow diverters, or fluid diodes. The apparatus may further comprise a flow detector or sensor. There may be a feedback loop between the flow detector or sensor and the flow controller whereby the flow detector or sensor is configured to produce a signal which is transmitted to the flow controller in order to control the flow rate of the solution(s) via the flow controller.

The apparatus may further comprise an inlet tube for connecting the fluid sample inlet port to a fluid sample source. It may also comprise an inlet tube for connecting the pH indicator inlet port to a source of pH indicator solution.

If desired, the microfluidic device and the apparatus described above can be used to online measure pH in an aqueous solution sample, such as a water sample from a swimming pool, a municipal water sample and an irrigation water sample. The person skilled in the art will appreciate that pH indicators are known and, provided response of the pH indicator results in a change in light absorbance, the change can be measured.

The present disclosure also provides a method of measuring pH in a fluid sample by using the microfluidic device described above.

2. Microfluidic Devices for Measuring More than One Parameter in a Fluid Sample

Also described herein is a microfluidic device for measuring more than one parameter in a fluid sample, the device comprising:

a sample microfluidic channel disposed on a solid substrate and configured to transfer the fluid sample to be analysed,

a first reagent microfluidic channel disposed on a solid substrate and configured to transfer a first reagent solution capable of reacting with a chemical substance in the fluid sample to produce a first parameter measurement solution having a response that is indicative of the first parameter in the fluid sample,

a second reagent microfluidic channel disposed on a solid substrate and configured to transfer a second reagent solution capable of reacting with a chemical substance in the fluid sample to produce a second parameter measurement solution having a response that is indicative of the second parameter in the fluid sample,

a mixing microfluidic channel disposed on a solid substrate and in fluid communication respectively with the sample microfluidic channel, the first reagent microfluidic channel and the second reagent microfluidic channel, which is configured to mix the fluid sample separately with the first reagent solution and the second reagent solution under conditions suitable for some of the first reagent to react with a chemical substance in the fluid sample to produce a first parameter measurement solution having a response that is indicative of the first parameter in the fluid sample and suitable for some of the second reagent to react with a chemical substance in the fluid sample to produce a second parameter measurement solution having a response that is indicative of the second parameter in the fluid sample, and

an optical reading window in fluid communication with an outlet of the mixing microfluidic channel, through which the first parameter and the second parameter can be optically measured.

The microfluidic device further comprises a waste microfluidic channel located downstream of the optical reading window.

It will be appreciated by the person skilled in the art that the microfluidic device described above can be used to measure at least two parameters of a fluid sample, for example, three parameters or four parameters. When the fluid sample is a water sample from a swimming pool, the parameters can be selected from an amount of active chlorine, an amount of total chlorine and pH. When the fluid sample is an irrigation water sample, the parameters can be alkalinity and pH. For the purpose of illustration, when there are three parameters to be measured, the microfluidic device may comprise a third reagent microfluidic channel disposed on a solid substrate and configured to transfer a third reagent solution capable of reacting with a chemical substance in the fluid sample to produce a third parameter measurement solution having a response that is indicative of the third parameter in the fluid sample, and the mixing microfluidic channel is also in fluid communication with the third reagent solution and is configured to mix the fluid sample with the third reagent solution suitable for some of the third reagent to react with a chemical substance in the fluid sample to produce a third parameter measurement solution having a response that is indicative of the third parameter in the fluid sample.

In some embodiments, the sample microfluidic channel, the first reagent microfluidic channel, the second reagent microfluidic channel, the third reagent microfluidic channel (optional), the mixing microfluidic channel, the optical reading window and the waste microfluidic channel are disposed on the same solid substrate. In some other embodiments, the mixing microfluidic channel, the optical reading window and, optionally, the waste microfluidic channel are disposed on a solid substrate different from the solid substrate(s) upon which the sample microfluidic channel, the first reagent microfluidic channel, the second reagent microfluidic channel and, optionally, the third reagent microfluidic channel are disposed.

The microfluidic device may also comprise a measuring chamber comprising the optical reading window and configured to separately receive the measurement solutions and through which the first parameter and the second parameter, etc. can be optically measured.

The solid substrate(s) used for the microfluidic device described herein can be made from glass, quartz, metal (e.g. stainless steel), ceramic, silicon, and polymers. Furthermore, the solid substrate(s) can be in the form of a chip. If there is more than one solid substrate, the two or more substrates may be connected to one another in series or parallel using suitable tubing and connectors, as is known in the art. For example, a through-hole can be used to connect an upper solid substrate and a lower solid substrate.

The chips can be thin, rectangular plates that are formed from a suitable material. Materials suitable for the manufacture of chips are known in the art and may be chosen based on considerations such as cost, inertness or reactivity toward fluids and other materials that will be in contact with the chip, etc. For instance, the chips may be formed from a transparent material which makes them suitable for forming the optical reading window. It can be contemplated that the chips may be formed from non-transparent materials and the optical reading window may be formed from a different transparent material. In certain embodiments, the substrate is a glass substrate. For example, Pyrex glass microfluidic chips may be suitable. Suitable polymeric substrates include polydimethylsiloxane (PDMS), polytetrafluoroethylene (PTFE), other perfluoropolyether (PFPE) based elastomers, polymethylmethacrylate (PMMA), silicone, and the like. The chips in the illustrated embodiments are rectangular in plan view but it is envisaged that they can be other shapes in plan view, such as circular, square, etc. The chips have a thickness adequate for maintaining the integrity of the microfluidic device.

The microfluidic channels may be formed on a solid substrate using any of the methods for forming fluid microchannel networks as are known in the art. For example, the chips can be fabricated using standard photolithographic and etching procedures including soft lithography techniques (e.g. see Shi J., et al., Applied Physics Letters 91, 153114 (2007); Chen Q., et al., Journal of Microelectromechanical Systems, 16, 1 193 (2007); or Duffy et al., Rapid Prototyping of Microfluidic Systems in Poly(dimethylsiloxane), Anal. Chem., 70 (23), 4974-4984 (1998)), such as near-field phase shift lithography, microtransfer moulding, solvent-assisted microcontact moulding, microcontact printing, and other lithographic microfabrication techniques employed in the semiconductor industry. Direct machining or forming techniques may also be used as suited to the particular chip. Such techniques may include hot embossing, cold stamping, injection moulding, direct mechanical milling, laser etching, chemical etching, reactive ion etching, physical and chemical vapour deposition, and plasma sputtering. The particular methods used will depend on the function of the particular microfluidic network, the materials used as well as ease and economy of production.

Variations of the size, shape and/or configuration of the microfluidic channels from those described are also envisaged. For example, the inlet microfluidic channels may be from 1 μm to 1000 μm in depth or width. The size of the microfluidic channels may also differ from one another in both dimensions.

Backflow of the fluid sample and the any one or more reagent during operation can be minimised through the flow resistance of any of the microfluidic channels. This can be done by forming a pressure gradient from a higher pressure inlet end of the microfluidic channels to a lower pressure outlet end of the microfluidic channels.

A pressure gradient could also be formed in the microfluidic channels using high-precision pumping and valving. For example, when all feeding pumps are stopped a pressure gradient will be formed within the device and this minimises backflow of the fluid sample and the pH indicator solution therein.

For the purpose of precise measurement, a diffusive mixing within the mixing microfluidic channel is desirable. To achieve mixing prior to measurement, the mixing microfluidic channel is configured so that the residence time from mixing the fluid sample separately with the first reagent solution, the second reagent solution and optionally the third reagent solution to arriving at the optical reading window is longer than the characteristic time for diffusive mixing. This can be achieved by appropriately choosing the cross-sections and the lengths of the microfluidic channels based on the total flow rate.

For the above purposes, the cross-section of the mixing microfluidic channel is greater in size than those of the sample microfluidic channel and the first reagent microfluidic channel, the second reagent microfluidic channel and, optionally, the third reagent microfluidic channel, and, optionally, the waste microfluidic channel. Considerations about its configuration will be discussed further under the section titled “Chip function”.

The microfluidic device may further comprise any one or more of:
a sample inlet port configured to receive the fluid sample to be analysed,
a first reagent inlet port configured to receive the first reagent solution capable of reacting with a chemical substance in the fluid sample to produce a first parameter measurement solution having a response that is indicative of the first parameter in the fluid sample,
a second reagent inlet port configured to receive the second reagent solution capable of reacting with a chemical substance in the fluid sample to produce a second parameter measurement solution having a response that is indicative of the second parameter in the fluid sample, and
a waste outlet port in fluid communication with the waste microfluidic channel and configured to allow the measurement solutions to exit the device.

The fluid sample inlet port and the reagent inlet ports can take any suitable form, such as an aperture or an opening.

The present disclosure also relates to an apparatus comprising a microfluidic device described above, which may further comprise any one or more of a pumping means, a light source and a detector. The sample and reagents may be transferred to the inlet ports and through the device under positive pressure provided by any suitable pump, by drawing the liquids through the device under vacuum, or by gravity feed. Devices for transferring liquids and gases to and through microfluidic networks are known in the art. For example, a syringe pump such as the ones from KD Scientific or a micro-syringe such as the ones under Gastight® from Hamilton Robotics. A light source can be configured to project light through the optical reading window. The light source to be used is dependent upon the chromogenic assay at hand. For example, narrow-band emission LEDs of various wavelengths including red, blue and green may be used to illuminate chromophores having certain absorbance bands. Diode lasers may also be used as a source of electromagnetic radiation. Broad-band sources such as a Tungsten lamp may be coupled with filters to select a wavelength used to probe a chromophore. Infra-red emitters may also be used. All of the foregoing may be used alone or in combination with each other, the choice dependent upon the assay/analyte to be detected. The detector can be employed to measure the absorbance of the solution reaching the optical reading window and may be a photodiode array spectrometer or a photodetector which is not wavelength selective. In the latter case, the incident light could be monochromatic. Examples for the detector include a custom-built micro-spectrophotometer based on an Olympus BH2-UMA frame and Ocean Optics Flame™ spectrophotometer.

The flow rates of the fluid sample and the first/second reagent solutions are independently controllable. The apparatus may further comprise at least one flow controller. The flow controller may include one or more valve, flow diverter, or fluid diode. The apparatus may further comprise a flow detector or sensor. There may be a feedback loop between the flow detector or sensor and the flow controller whereby the flow detector or sensor is configured to produce a signal which is transmitted to the flow controller in order to control the flow rate of the solution(s) via the flow controller.

The apparatus may further comprise an inlet tube for connecting the fluid sample inlet port to a fluid sample source. It may also comprise an inlet tube respectively for connecting the first reagent inlet port and the second reagent inlet port to a source of the first reagent solution and the second reagent solution.

If desired, the microfluidic device described above can be used to do online measurement. The fluid sample may be an aqueous solution sample, such as a water sample from a swimming pool, a municipal water sample or an irrigation water sample. Reagents for doing a measurement may be appropriately chosen by the person skilled in the art, provided a response that is indicative of a parameter in the fluid sample results in a change in light absorbance which can be optically measured.

The present disclosure also relates to a method of measuring more than one parameter in a fluid sample by using the microfluidic device described above. The details regarding the method will be discussed later.

3. Microfluidic Devices for Measuring an Amount of Active Chlorine, an Amount of Total Chlorine and pH in a Fluid Sample

Also described herein is a microfluidic device for measuring an amount of active chlorine, an amount of total chlorine and pH in a fluid sample, the device comprising:

a sample microfluidic channel disposed on a solid substrate and configured to transfer the fluid sample to be analysed,

a first reagent microfluidic channel disposed on a solid substrate and configured to transfer a first indicator dye solution capable of reacting with any active chlorine in the fluid sample to produce an active chlorine measurement solution having a reduced indicator dye concentration that is indicative of the amount of active chlorine in the fluid sample,

a second reagent microfluidic channel disposed on a solid substrate and configured to transfer a second indicator dye solution capable of reacting with any total chlorine in the fluid sample to produce a total chlorine measurement solution having a reduced indicator dye concentration that is indicative of the amount of total chlorine in the fluid sample,

a third reagent microfluidic channel disposed on a solid substrate and configured to transfer a pH indicator solution capable of responding to pH in the fluid sample to produce a pH measurement solution having a response indicative of the pH,

a mixing microfluidic channel disposed on a solid substrate and in fluid communication respectively with the sample microfluidic channel, the first reagent microfluidic channel, the second reagent microfluidic channel and the third reagent microfluidic channel, which is configured to mix the fluid sample separately with the first indicator dye solution, the second indicator dye solution and the pH indicator solution under conditions suitable for some of the indicator dye in the first indicator dye solution to react with any active chlorine in the fluid sample to produce an active chlorine measurement solution having a reduced indicator dye concentration that is indicative of the amount of active chlorine in the fluid sample, suitable for some of the indicator dye in the second indicator dye solution to react with any total chlorine in the fluid sample to produce a total chlorine measurement solution having a reduced indicator dye concentration that is indicative of the amount of total chlorine in the fluid sample, and suitable for the pH indicator to respond to pH in the fluid sample to produce a pH measurement solution having a response indicative of the pH, and

an optical reading window located downstream of the mixing microfluidic channel, through which the amount of active chlorine, the amount of total chlorine and pH in the fluid sample can be optically measured.

The microfluidic device also comprises a waste microfluidic channel located downstream of the optical reading window.

The microfluidic device may also comprise a measuring chamber comprising the optical reading window and configured to separately receive the measurement solutions and through which the amount of active chlorine, the amount of total chlorine, and pH in the fluid sample can be measured optically.

The solid substrate(s) used for the microfluidic device described herein can be made from glass, quartz, metal (e.g. stainless steel), ceramic, silicon, and polymers. Furthermore, the solid substrate(s) can be in the form of a chip. If there is more than one solid substrate, the two or more substrates may be connected to one another in series or parallel using suitable tubing and connectors, as is known in the art. For example, a through-hole can be used to connect an upper solid substrate and a lower solid substrate.

The chips can be thin, rectangular plates that are formed from a suitable material. Materials suitable for the manufacture of chips are known in the art and may be chosen based on considerations such as cost, inertness or reactivity toward fluids and other materials that will be in contact with the chip, etc. For instance, the chips may be formed from a transparent material which makes them suitable for forming the optical reading window. It can be contemplated that the chips may be formed from non-transparent materials and the optical reading window may be formed from a different transparent material. In certain embodiments, the substrate is a glass substrate. For example, Pyrex glass microfluidic chips may be suitable. Suitable polymeric substrates include polydimethylsiloxane (PDMS), polytetrafluoroethylene (PTFE), other perfluoropolyether (PFPE) based elastomers, polymethylmethacrylate (PMMA), silicone, and the like. The chips in the illustrated embodiments are rectangular in plan view but it is envisaged that they can be other shapes in plan view, such as circular, square, etc. The chips have a thickness adequate for maintaining the integrity of the microfluidic device.

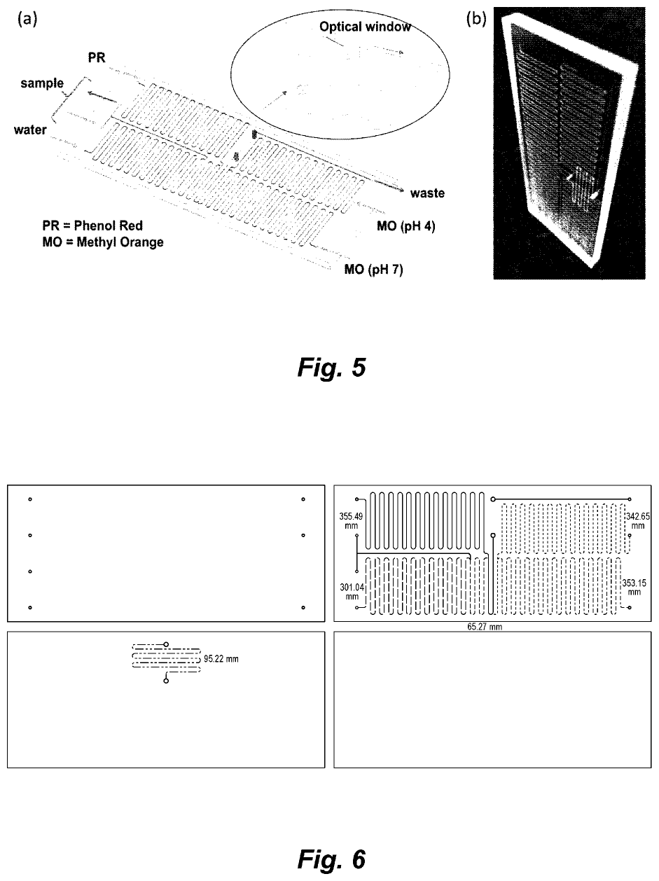

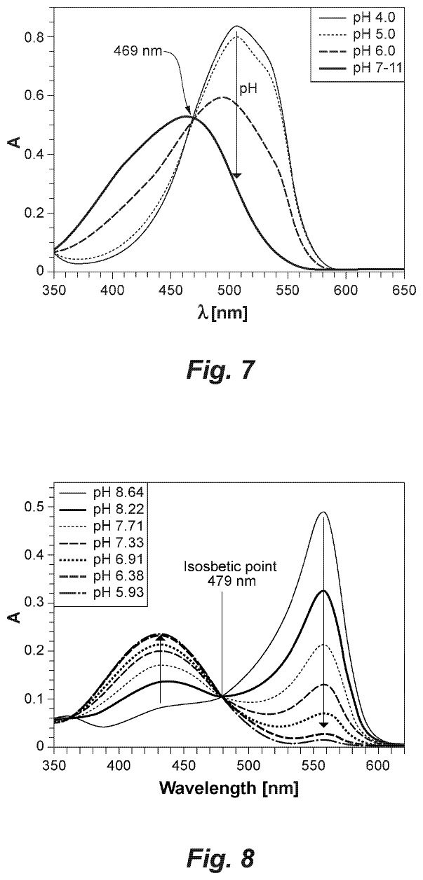

FIGS. 4 and 5 show one embodiment of a microfluidic device for measuring an amount of active chlorine, an amount of total chlorine and pH in a fluid sample. The microfluidic device comprises:
a sample microfluidic channel (designated as “sample”) disposed on the upper solid substrate and configured to transfer the fluid sample to be analysed,
a first reagent microfluidic channel (designated as “MO (pH 7)”) disposed on the upper solid substrate and configured to transfer a first indicator dye solution capable of reacting with any active chlorine in the fluid sample to produce an active chlorine measurement solution having a reduced indicator dye concentration that is indicative of the amount of active chlorine in the fluid sample,
a second reagent microfluidic channel (designated as “MO (pH 4)”) disposed on the upper solid substrate and configured to transfer a second indicator dye solution capable of reacting with any total chlorine in the fluid sample to produce a total chlorine measurement solution having a reduced indicator dye concentration that is indicative of the amount of total chlorine in the fluid sample,
a third reagent microfluidic channel (designated as “PR”) disposed on the upper solid substrate and configured to transfer a pH indicator solution capable of responding to pH in the fluid sample to produce a pH measurement solution having a response indicative of the pH,
a mixing microfluidic channel (shown as a red line in the inset) disposed on the lower solid substrate and in fluid communication respectively with the sample microfluidic channel, the first reagent microfluidic channel, the second reagent microfluidic channel, and the third reagent microfluidic channel, which is configured to mix the fluid sample separately with the first indicator dye solution, the second indicator dye solution and the pH indicator solution under conditions suitable for some of the indicator dye in the first indicator dye solution to react with any active chlorine in the fluid sample to produce an active chlorine measurement solution having a reduced indicator dye concentration that is indicative of the amount of active chlorine in the fluid sample, suitable for some of the indicator dye in the second indicator dye solution to react with any total chlorine in the fluid sample to produce a total chlorine measurement solution having a reduced indicator dye concentration that is indicative of the amount of total chlorine in the fluid sample, and suitable for the pH indicator to respond to pH in the fluid sample to produce a pH measurement solution having a response indicative of the pH, and
an optical reading window (designated as “optical window”) located downstream of the mixing microfluidic channel on the lower solid substrate, through which the amount of active chlorine, the amount of total chlorine and pH in the fluid sample can be optically measured, and
a waste microfluidic channel (designated as “waste”) located downstream of the optical reading window on the lower solid substrate,
   wherein the sample microfluidic channel, the first reagent microfluidic channel, the second reagent microfluidic channel, the third reagent microfluidic channel, and the mixing microfluidic channel are serpentine in form.

In the illustrated device, the sample channel connects to a T-junction. One path goes into the device for analysis and the other to an additional outlet. The additional outlet allows flushing of the sample to avoid waste of reagents while the sample inlet tube volume is flushed through. For example, prior to a measurement, the sample will be flushed through to the additional sample waste outlet until the fresh sample is present in the feed tubing and the sample microchannel (upstream of the T-junction). At this time, the waste outlet is closed (using a suitable valve), which will direct the sample stream into the device for measurement. The process would repeat for the next sample measurement. This is a semicontinuous operation to save reagents etc. The water channel offers the chance to flush the system to prevent scale, biofilms, bacteria, etc building up in between measurements. In addition, a water stream offers the chance to do a baseline check between measurements (or from time to time) to make certain that drift or fouling is not giving false readings. The water channel works in the same way as any other reagent/sample stream.

Despite the above embodiment shown in FIGS. 4 and 5, it will be appreciated by the person skilled in the art that the microfluidic device can be configured so that the sample microfluidic channel, the first reagent microfluidic channel, the second reagent microfluidic channel, the third reagent microfluidic channel, the mixing microfluidic channel and the optical reading window are disposed on the same solid substrate.

More specifically, as best seen in FIG. 6, the microfluidic device is a multilayer microfluidic device comprising a number of solid substrates in the form of a chip, i.e. first and second outer chips and first and second intermediate chips, and wherein the sample microfluidic channel, the first reagent microfluidic channel, the second reagent microfluidic channel, and the third reagent microfluidic channel are disposed on the first intermediate chip, and the mixing microfluidic channel and the optical reading window are disposed on the second intermediate chip.

As described earlier, backflow of the fluid sample and the reagents during operation can be minimised through the flow resistance of any of the microfluidic channels and/or using high-precision pumping and valving.

For the purpose of precise measurement, a diffusive mixing within the mixing microfluidic channel is desirable. To achieve mixing prior to measurement of pH, the mixing microfluidic channel is configured so that the residence time from mixing the fluid sample separately with the first indicator dye solution, the second indicator dye solution and the pH indicator solution to arriving at the optical reading window is longer than the characteristic time for diffusive mixing. This can be achieved by appropriately choosing the cross-sections and the lengths of the microfluidic channels based on the total flow rate.

For the above purposes, the cross-section of the mixing microfluidic channel is greater in size than those of the sample microfluidic channel and the first/second/third reagent microfluidic channels and, if present, the waste microfluidic channel. For the embodiment shown in FIG. 1, the cross-sections of the sample microfluidic channel, the pH indicator microfluidic channel, and the waste microfluidic channel are configured to be 103 μm×214 μm, and the cross-section of the mixing microfluidic channel is configured to be 117 μm×245 μm. This configuration can allow completion of diffusive mixing before measurement of pH and prevent backflow of the fluid sample and the reagents during operation. In the illustrated embodiments, the mixing microfluidic channel has a length of about 95 mm Considerations about its configuration will be discussed further under the section titled “Chip function”. The optical cell has a 2.2 mm path length. It is readily understood that a longer path length will inevitably increase the sensitivity.

The microfluidic device may further comprise any one or more of:
a sample inlet port configured to receive the fluid sample to be analysed,
a first indicator dye inlet port configured to receive the first indicator dye solution capable of reacting with any active chlorine in the fluid sample to produce an active chlorine measurement solution having a reduced indicator dye concentration that is indicative of the amount of active chlorine in the fluid sample,
a second indicator dye inlet port configured to receive the second indicator dye solution capable of reacting with any total chlorine in the fluid sample to produce a total chlorine measurement solution having a reduced indicator dye concentration that is indicative of the amount of total chlorine in the fluid sample,
a pH indicator inlet port configured to receive the pH indicator solution containing a pH indicator capable of responding to pH in the fluid sample to produce a pH measurement solution having a response indicative of the pH, and
a waste outlet port in fluid communication with the waste microfluidic channel and configured to allow the measurement solution to exit the device.

The fluid sample inlet port and other inlet ports can take any suitable form. For the embodiment shown in FIGS. 4 to 6, the fluid sample inlet port, the first indicator dye inlet port, the second indicator dye inlet port, and the pH indicator solution inlet port can be formed in the first outer chip. In the illustrated embodiment, the inlet ports are in the form of apertures or openings in the first outer chip.

The present disclosure also relates to an apparatus comprising a microfluidic device described above, which may further comprise any one or more of a pumping means, a light source and a detector. The sample and reagents may be transferred to the inlet ports and through the device under positive pressure provided by any suitable pump, by drawing the liquids through the device under vacuum, or by gravity feed. Devices for transferring liquids and gases to and through microfluidic networks are known in the art. For example, a syringe pump such as the ones from KD Scientific or a micro-syringe such as the ones under Gastight® from Hamilton Robotics. A light source can be configured to project light through the optical reading window. The light source to be used is dependent upon the chromogenic assay at hand. For example, narrow-band emission LEDs of various wavelengths including red, blue and green may be used to illuminate chromophores having certain absorbance bands. Diode lasers may also be used as a source of electromagnetic radiation. Broad-band sources such as a Tungsten lamp may be coupled with filters to select wavelength used to probe a chromophore. Infra-red emitters may also be used. All of the foregoing may be used alone or in combination with each other, the choice dependent upon the assay/analyte to be detected. The detector can be employed to measure the absorbance of the solution reaching the optical reading window and may be a photodiode array spectrometer or a photodetector which is not wavelength selective. In the latter case, the incident light could be monochromatic. Examples for the detector include a custom-built micro-spectrophotometer based on an Olympus BH2-UMA frame and Ocean Optics Flame™ spectrophotometer.

The flow rates of the fluid sample, the first/second indicator dye solution and the pH indicator solution are independently controllable. The apparatus may further comprise at least one flow controller. The flow controller may include one or more valves, flow diverters, or fluid diodes. The apparatus may further comprise a flow detector or sensor. There may be a feedback loop between the flow detector or sensor and the flow controller whereby the flow detector or sensor is configured to produce a signal which is transmitted to the flow controller in order to control the flow rate of the solution(s) via the flow controller.

The apparatus may further comprise an inlet tube for connecting the fluid sample inlet port to a fluid sample source. It may also comprise an inlet tube respectively for connecting the first indicator dye inlet port, the second indicator dye inlet port, and/or the pH indicator inlet port to a source of the first indicator dye solution, the second indicator dye solution, and/or the pH indicator solution.

If desired, the microfluidic device and the apparatus described above can be used to online measure an amount of active chlorine, an amount of total chlorine and pH in a fluid sample, for example, an aqueous solution sample. The aqueous solution sample may be a water sample from a swimming pool, a municipal water sample or an irrigation water sample. The person skilled in the art will appreciate that the indicator dyes for detecting active chlorine and total chlorine are known and the pH indicators are also known, provided response of the indicator dye(s) and the pH indicator results in a change in light absorbance, the change can be measured.

The present disclosure also relates to a method of measuring an amount of active chlorine, an amount of total chlorine and pH in a fluid sample by using the microfluidic device described above. The details regarding the method will be discussed later.

Although the following more detailed description is made with reference to the case wherein the fluid sample is from a swimming pool and the measurement is for an amount of active chlorine, an amount of total chlorine and pH, it is not intended to limit the scope of the embodiments, as claimed, but is merely representative of example embodiments.

Materials

Swimming pool water samples were collected from 12 outdoor pools (9 domestic; 2 indoor public; 1 outdoor public) located in metropolitan Adelaide, Australia. The three public pools are used by many people on a daily basis. The sample IDs are anonymous (numbered), as the identity and location of the pools and donors is unimportant in this study. Samples (500 mL) were collected and stored at 4° C. until use.

The indicator dye used for detecting active chlorine and total chlorine may include organic azo dyes, organic amine dyes, and thioninium dyes. Exemplary organic azo dyes include sodium 4-[(4-dimethylamino)phenyldiazenyl]benzenesulfonate (i.e. methyl orange). Exemplary organic amine dyes include DPD. Exemplary thioninium dyes include methylene blue. The pH indicator used herein may be selected from thymol blue, methyl yellow, phenol red, congo red, methyl orange, methyl red, neutral red and alizarine yellow R.

The concentration of the indicator dye in the solution containing an indicator dye may be from about 1 ppm to about 1000 ppm. In the case of methyl orange, the concentration may be selected from the group consisting of 20, 30, 40, 50 and 100 ppm.

There are several advantages of MO for the continuous monitoring of chlorine: the reagent is stable indefinitely, MO is a readily available, interference from iron, nitrite and chloramines is negligible²⁰ and the chloramine (total chlorine) detection can be accelerated with the addition of bromide ions (Br⁻). In the illustrated embodiments, the indicator dye is an organic azo dye, such as sodium 4-[(4-dimethylamino)phenyldiazenyl]benzenesulfonate (i.e. methyl orange or “MO”). MO is an organic azo-dye and a pH-indicator used for strong acid—strong base titrations. It has a pKa (303K) value of 3.5 and is red for pH<3.1 and yellow for pH>4.4. MO is bleached in the presence of chlorine solution. This decolorization can be detected optically.

The temperature influence on the absorbance of MO was found to be negligible for 10-30° C.^{20, 22} The spectrum of methyl orange is dependent on the pH of the sample and the pH-influence is greatly pronounced near the pKa-value of MO. There are three points between 250-600 nm where the absorbance of MO does not depend on pH. These points are called isosbestic points. The isosbestic point at 469 nm is most suitable as a detection wavelength for the bleaching reaction of MO with chlorine.

Sodium hypochlorite containing 4.00-4.99% available chlorine, phenol red (ACS grade) and starch (ACS grade) were purchased from Sigma Aldrich and used without further purification. Methyl orange, anhydrous citric acid, potassium iodide, sodium hydrogen bicarbonate and sodium thiosulfate were obtained from Chem Supply as analytical grade materials. Glacial acetic acid (Chem Supply, 80%), sodium bromide (Scharlau, extra pure) and all other chemicals were used as obtained from the supplier. Deionised water (Milli-Q® Advantage A10 Water Purification System, Merck Millipore) was used to dilute all samples and dye solutions to the required concentrations.

For the active chlorine analysis, 100 ppm MO stock solution containing 1000 ppm NaBr was used. The solution is further referred as MO (pH 7). For the total chlorine analysis, buffered, acidified 100 ppm MO stock solution containing 4000 ppm NaBr was used. The solution was prepared from 90 ml of 63 mM citrate buffer pH=4, 400 mg NaBr and 10 ml of 1000 ppm aqueous solution of MO. In the following discussion, the solution is referred as MO (pH 4).

Calibration lines for active and total chlorine were obtained by measuring sodium hypochlorite solutions containing 0-10 ppm of hypochlorite. Procedures for the measurements were the same as described below for real samples. The calibration solutions were prepared by dissolving aliquots of 470 ppm NaClO in 50 ppm NaHCO₃. The 470 ppm NaOCl solution was prepared by dilution of commercially available sodium hypochlorite containing 4.00-4.99% available chlorine and was standardized using iodometric method.²¹

For the pH analysis, the phenol red solution was prepared by modifying a commercially available pH indicator dye solution (HYCLOR® pH Phenol Red), containing 4.5% chlorine quencher. The concentration of PR was optimised for use in the chip by diluting a 2 mL pH indicator solution with 398 mL DI water and adding 16 mg solid PR (Sigma Aldrich). To help dissolve the solid PR, one drop of 1M NaOH was added. A calculation of the concentration of PR in the prepared solution (pH 6.4) was 41.2 mg/L phenol red, based on the Beer-Lambert analysis.

Calibration points for pH method were prepared by adjustment of a randomly chosen swimming pool sample. Aliquots (20 mL) of the sample were adjusted to the required pH (monitored using a pH meter) by addition of several drops of 0.5 M NaOH or 0.5 M HCl and measured as described below for real samples.

Chip Design and Preparation

Here, a lab-on-a-chip sensor capable of measuring three parameters is investigated, see FIG. 1. The use of photometric methods demands clear windows and a suitable path length for detection. In relation to a microfluidic device for measuring an amount of active chlorine, an amount of total chlorine and pH in a fluid sample, a four-layer thermally bonded glass chip was used. The upper and lower layers provided the clear glass windows for the photometric sensing and the two middle layers contained through-holes (with one used as the optical cell, FIG. 5(a) inset), and the microchannel network. The design of the bonded chip is shown in FIG. 5(a), with an inset showing the details of the optical window and the surrounding channels.

The chip uses photometric analysis of methyl orange and phenol red solutions in a 2.2 mm path length optical cell in a borosilicate glass chip. The estimated reagent use over a season (3 months) is approximately 33 mL, depending on operational protocols.

The chip was prepared in borosilicate glass (Borofloat 33) via Cr/Au-photoresist masking followed by wet etching using 50% hydrofluoric acid. The through-holes (optical cell and inlet/outlet ports) were laser machined. Masking materials were removed by cerium ammonium nitrate solution, iodine/iodide solution, and acetone for Cr, Au, and photoresist, respectively. Thermal bonding was achieved at 630° C. and 1.5 kPa. A custom chip holder was prepared in polymethylmethacrylate (PMMA) to allow interfacing with FEP tubing.

The lengths of the serpentine channels prevent backflow of the reagent and sample. These channels, and the outlet channel beyond the optical cell, have a cross-section of 103 μm×214 μm. The channel in the lower layer of the chip (channel marked in red) immediately before the optical cell, allows diffusive mixing before detection of MO or PR in the optical cell, FIG. 5(a). This channel has a cross-section of 117 μm×245 μm, and is 9.4 cm long. The optical cell has a path length of 2.2 mm and a diameter of 1 mm, ie 17 μL volume.

Experiments

Off-Chip Experiments

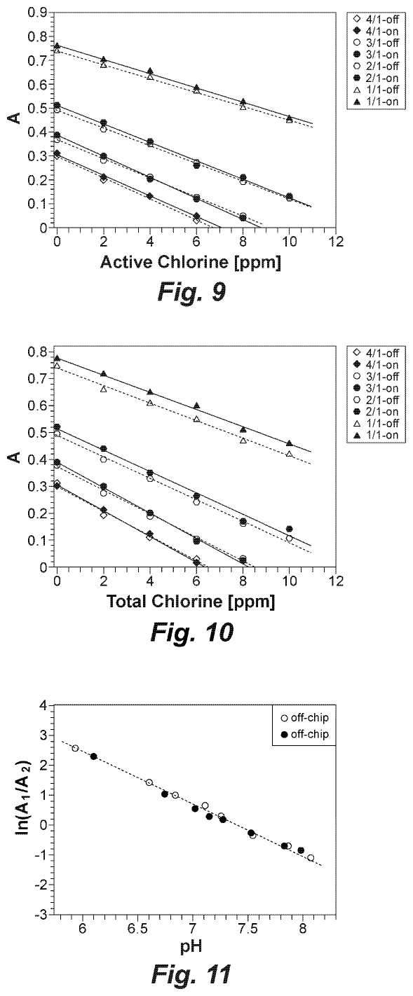

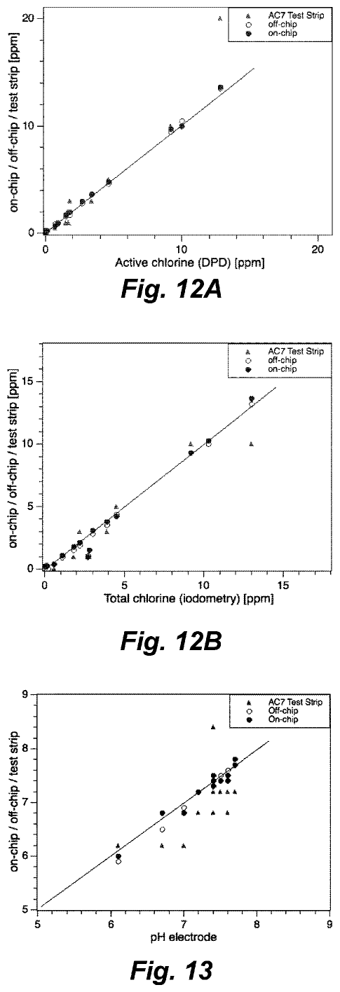

All experiments in the bulk (off-chip) were conducted at room temperature (r.t.). The collected swimming pool samples were stored at 4° C. and allowed to reach r.t. prior to their analysis. All dye solutions were kept at r.t. in the dark. Off-chip UV-Vis experiments were conducted in a 2 mm quartz cuvette (Ocean Optics, QE65000). For chlorine analyses, the detection wavelength was set at the isosbestic point of MO (469 nm)²³ to eliminate the bathochromic shift in the acidic pH regime (FIG. 9). The methyl orange solution was pipetted into the cuvette before the sample was added and immediately mixed vigorously. For a 1:1 mixing ratio, 200 μl of sample and 200 μl of MO was used. For 2:1 mixing ratio, 400 μl of sample and 200 μl of MO was used. For 3:1 mixing ratio, 150 μl of sample and 50 μl of MO was used. For 4:1 mixing ratio, 200 μl of sample and 50 μl of MO was used.

The pH analysis in the bulk was conducted in a 2 mm quartz cuvette on the Ocean Optics spectrometer by mixing the sample and the PR stock solution in 1:1 ratio (200 μl of sample and 200 μl of PR). Absorbance spectra for phenol red are given in FIG. 8. The natural logarithm of the relative absorbance A1/A2, 432/560 nm, was recorded. The pH was measured with pH meter OAKTON pH 2700 using an ORION 8220BNWP pH micro electrode (Thermo Fisher Scientific). The probe was calibrated against pH 4, 7 and 10 buffer solutions. For the iodometric method, we used the international standard procedure²¹, modified for low concentrations of chlorine in pool water. Our procedure follows the method for available chlorine, the only difference is use of 0.5 mM sodium thiosulfate instead of 0.1 M. The solution was prepared by diluting 2.5 mL of the 0.1 M Na₂S₂O₃ standard to 500 mL with deionised, freshly boiled, then cooled water. When standardizing against potassium iodate our sodium thiosulfate solution was found to be 0.47 mM. Potassium iodide (2-3 g) and acetic acid (10 mL) were dissolved in 50 mL of water in a 250 mL Erlenmeyer flask and 50 mL aliquot of the sample was added. The sample was then titrated with the Na₂S₂O₃ solution until the iodine colour was nearly gone. After adding 1 mL of the starch solution²¹, the end point of the further titration with Na₂S₂O₃ was found, when the blue colour of the starch disappeared. The titration was repeated three times and the average end point was used for the calculation of the total chlorine levels.

Universal test strips (AquaCheck® AC7, HACH®) were used by submerging the strips into the sample solution and matching the colour with the colour chart after a 15 second reaction time.

The DPD powder (5 mL powder pillow, HACH® Permachem Reagents) was added to 5 mL sample solution and shaken for 20 seconds. Then, the absorbance of the DPD solution at 530 nm was immediately recorded on the UV-Vis spectrophotometer (Ocean Optics) in a 10 mm quartz cuvette. The concentrations of the active chlorine levels were calculated from the calibration line. Calibration was found to be linear between 0-5 ppm. To analyse more concentrated samples, 1 mL of sample was diluted with 4 mL of water and then treated as described above. The result was multiplied by the dilution factor to obtain original concentration of the sample.

On-Chip Experiments

On-chip experiments were conducted using the same reagent solutions and conditions as used for the off-chip experiments. Samples and reagents were pumped using precision syringe pumps (KD Scientific) and 1 mL micro-syringes (Gastight® Instruments Syringes, HAMILTON). The streams merged upstream of the optical cell, mixed by diffusion, and then were measured through the optical window. Proof-of-principle experiments were successfully conducted using LEDs and photodiodes, however, the results presented here were obtained using a custom-built micro-spectrophotometer, based on an Olympus BH2-UMA frame.²⁶ This allowed the full spectrum (200-1000 nm) to be collected and inspected for potential interferences or anomalies (e.g. in real pool samples). In addition, the micro-spectrophotometer has an in-built digital camera that allows the user to visualise the channels and check for errors due to fouling or air bubbles.

TABLE 1
On-chip measurement protocol

| | | FLOW RATE μL/MIN) | | | |
|---|---|---|---|---|---|
| | TIME | | MO | MO | |
| PROTOCOL STEP | (MIN) | S | PH7 | PH4 | PR |
| SAMPLE FLUSH | 2 | 50 | 0 | 0 | 0 |
| ACTIVE CL (<8 PPM)* | 6 | 7.5 | 2.5 | 0 | 0 |
| TOTAL CL (<8 PPM)* | 6 | 7.5 | 0 | 2.5 | 0 |
| PH | 6 | 5 | 0 | 0 | 5 |

*FOR >8 PPM, SAMPLE/MO WAS 1/1.

On-chip measurements were collected using the protocol in Table 1. First, the sample channel was flushed with 100 μl of sample. Then, for active chlorine analysis, MO (pH 7) was pumped together with the sample at the required flow rate ratio. The overall flow rate was 10 μl/minute in all cases. For analysis of real samples, mixing ratio 1:3 was used for samples containing less than 8 ppm active chlorine, a 1:1 mixing ratio was used when the concentration was higher. Changes in absorbance at 469 nm and 650 nm (background) were observed using the OceanView strip chart. After stabilizing of signals (<2 minutes), the absorbance was recorded for 4 minutes. The absorbance at 650 nm (background) was subtracted from that at 469 nm. The average value was used for calculating the concentration to minimize the influence of any transient instabilities.

After active chlorine analysis, MO (pH 7) pump was turned off and MO (pH 4) pump was turned on for total chlorine analysis. The protocol was the same as for active chlorine analysis described above.

After total chlorine analysis, the MO (pH 4) pump was turned off and PR turned on. Mixing ratio was 1:1 (5 μl/minute sample and 5 μl/minute PR). Absorbances at 432, 560 and 650 nm were observed in OceanView strip chart. After stabilizing of signals, absorbances were recorded for 4 minutes. Logarithm of relative peak intensity was calculated, ln((A₄₃₂−A₆₅₀)/(A₅₆₀−A₆₅₀)), and the average value used to calculate the pH.

Results and Discussions

Chip Function

First, the physical parameters of the device and their impact on the chip operation are considered. The three critical parameters are pressure drop, residence time, and diffusion time. The flow in the chip must meet two key conditions: (1) the flow resistance in the sample and reagent inlet streams should be large enough to minimise backflow during operation and (2) the residence time from merging of sample and reagent streams to arriving at the optical cell should be longer than the characteristic time for diffusive mixing.

The hydrodynamic pressure drop pᵢ for a given channel segment i is given by the Hagen-Poiseuille equation:

$${p}_{i} = \frac{8 μ {L}_{i}}{π {r}_{i}^{4}} {Q}_{i}$$

where μ is the dynamic viscosity of the fluid, Lᵢ is channel length, Qᵢ is the flow rate, and rᵢ is the hydraulic radius²⁵ that is defined by xy/(x+y) for a rectangular channel cross-section, where x and y are the channel width and channel depth, respectively. If we consider the chip design in FIG. 1, the sample and reagent flow separately through serpentine channels (s) and together through the mixing (m), and waste (w) channels; Qₘ=Q_{w}=Qₛ+Qᵣ. The chip is designed to operate at different sample/reagent flow ratios, R=Qs/Qr, to tune the sensitivity to chlorine. In principle, pₛ>pₘ'p_{w} to avoid risk of backflow. For the present design, pₛ˜3-5 kPa, pₘ˜1.2 kPa, and p_{w}˜0.5 kPa, thus meeting this condition.

The residence time tₘ in the mixing channel is given by Vₘ/Qₘ, where Vₘ is the volume of the channel. The characteristic time for diffusive mixing (perpendicular to the laminar flow) can be estimated by t=x²/D, where x and D (˜10⁻⁹ m²/second) are the channel width and diffusion coefficient (MO or PR), respectively. To achieve mixing prior to the optical cell, tₘ>t. Based on the total flow rate used (10 μL/min), the residence time in the mixing channel is approx. 20 seconds, which is greater than the calculated diffusion time (14 seconds). This analysis is conservative as it ignores the actual flow profile of the two laminar streams and the residence time in the first through-hole, which would accelerate mixing. In practice, we observe complete mixing and conclude that the chip design successfully delivers a homogeneous stream to the optical cell.

The chip design and operational protocol (Table 1) uses micro-volume samples per measurement. The chip requires 220 μL sample, 15 μL MO (pH 7), 15 tit MO (pH 4), and 30 μL PR for a complete measurement cycle, or less than 35 mL (8.2 mL MO (pH 7); 8.2 mL MO (pH 4); 16.4 mL PR) of each reagent for 3 months operation at 6 cycles per day.

Active Chlorine

Photometric analysis of bleaching of MO by active chlorine has many practical advantages for our application. MO is widely available, has a low toxicity, and is highly stable. In addition, the proposed method has negligible interference from iron, manganese and nitrite, which are known to interfere with other methods for determining active chlorine. Experiments revealed >6 months stability under ambient temperature, low temperature (4° C.), direct sunlight, and atmospheric oxygen.

Literature²⁴ suggests that analysis of active chlorine using this method should be carried out at acidic pH. In this study, we have modified the method to avoid interference from bound chlorine (chloramines). We use unbuffered MO solution containing NaBr. Due to the high buffering capacity of swimming pool samples, the pH of the MO-sample mixture remains close to neutral (pH 6 to 8). In this pH range, active chlorine reacts with bromide to form active bromine, which quantitatively reacts with MO, and bound chlorine does not interfere. Optimization of the bromide concentration revealed that 1000 ppm was most suitable for the application.

The absorbance of methyl orange strongly depends on the pH of the solution.²³ As shown in FIG. 2, acidification of the dye solution in bulk showed bathochromic shifts in the MO spectra, whereas it remained unchanged under alkaline conditions. In our studies, the detection wavelength was set at the isosbestic point of methyl orange at 469 nm to negate minor fluctuations in the pH during the analysis, despite the pH being buffered in swimming pool water. The following results report “on-chip” and “off-chip” (bulk) analyses of active chlorine using the method described above. The reaction between the active chlorine sample (S) and 100 ppm MO was carried out using a S/MO volume ratio of 1:1, 2:1, 3:1, and 4:1 (open symbols, FIG. 9). On-chip experiments were carried out using the same ratios, which were achieved by varying the relative flow rates at a fixed total flow 10 μL/min. This total flow rate allowed sufficient mixing time (16 seconds) before entering the optical cell, see Chip Function section. Good agreement between the on-chip and off-chip methods was obtained, with improved agreement with increasing S/MO ratio. The reason for this is unclear. However, we found that higher S/MO ratios (3:1) are well-suited to chlorine concentrations <8 ppm (as typically found in swimming pools) and has practical advantages, including storage of lower volumes of reagent.

The ability to tune the flow ratio enables the sensitivity and range of measurements to be varied, depending on the sample. The lower ratio (1:1) is therefore only used for overdosed pools (>8 ppm), as shown later.

Total Chlorine

Active and bound chlorine—collectively ‘total’ chlorine—react with MO at low pH in the presence of NaBr. In this study, we prepared a MO solution (pH 4-4.5, citrate buffer; 4000 ppm NaBr) that achieves a fast and quantitative reaction. FIG. 10 shows the calibrations at different S/MO at pH 4 for on-chip and off-chip experiments. A comparative analysis of the absorbance changes and dilution effects as a function of the added chlorine showed an adjustment to the reaction stoichiometry, similar to previous studies on active chlorine. The highest S/MO ratio (4:1) led to the highest sensitivity but only over a small concentration range (0-6 ppm). Therefore, the S/MO ratio of 3:1 was preferred for measurements of real pool samples (<8 ppm).

pH Analysis

The photometric pH analysis exploits the pH-sensitive spectrum of the acid-base indicator phenol red (PR). PR has two distinct absorbance bands at 430 nm (A432) and 560 nm (A560) in the visible range, which change dramatically between pH 6 (yellow) and 8 (pink/red) and are separated by an isosbestic point at 479 nm, FIG. 8.²⁶ PR is not currently used in an online pH monitor, possibly due to reagent consumption. In a microfluidic device, the reagent consumption can be small, even when used for many measurements over long periods of time (as discussed here).

FIG. 11 shows the natural logarithm of the relative peak intensities (A₄₃₂/A₅₆₀) in the pH range 6.0-8.5. These calibration samples were prepared using an outdoor, domestic swimming pool sample (initial pH 7.3), which was spiked with 0.5 M HCl or 0.5 M NaOH solutions to tune the pH over the calibration range. The S/PR volume (off-chip) or flow (on-chip) ratio was 1:1. Good agreement between the photometric method and a pH electrode was obtained. When the pH was below 6 or above 8.5, the linear relationship was not observed. This is due to either A₄₃₂ or A₅₆₀ approaching zero. However, these limits would appear in the pH sensor as values that exceed high or low limits, alerting the user to the need for pool maintenance.

Testing Swimming Pools

To validate the microfluidic swimming pool sensor, we tested samples obtained from 12 swimming pools (9 domestic; 2 indoor public; 1 outdoor public). Samples 11 and 12 were collected and measured on multiple occasions. Every sample had its own ambient situation and sanitation history thus representing a realistic challenge for the sensor. Examples included a recently purchased property, high organic load (e.g. leaf and animal litter), frequent public use, indoor pools, and various chlorination methods. Owners and operators were not asked to provide any information about their pool prior to analysis. All samples were tested for active chlorine, total chlorine, and pH using photometric (on-chip and off-chip) and other methods discussed below.

Commercially available universal test-strips AC7® from AquaCheck (HACH) were used to read the pH, active and total chlorine levels from the colour table. For greater accuracy, commercial DPD1® (N,N′-diethyl-p-phenylenediamine) powder pillows were used (bulk analysis) for active chlorine. Total chlorine was analysed by iodometric titration. Measurement of pH was carried out using a precise laboratory-grade pH electrode. For details of these measurement methods, see FIGS. 9 and 10 which compare the on-chip and off-chip results with the benchmark results for all 12 samples. In general, there is good agreement between the on-chip and benchmark results. However, for AC7® test strips, the large concentration steps in the colour chart (active chlorine: 0, 0.5, 1, 3, 5, 10, 20 ppm) led to significant scatter in these measurements.

The on-chip analysis of active chlorine was in good agreement with the DPD1® benchmark. For total chlorine, all samples except 3 and 4 agreed with the iodometric titration. Iodometry is non-specific for chlorine, with manganese, iron, and other oxidants known to interfere. Inductively coupled plasma mass spectroscopy (ICP-MS) reported high average concentrations (>100 ppm) of Na and Ca, as expected, and low concentrations of K (5-16 ppm), B (0-7 ppm), Mg (4-34 ppm) and Si (<5 ppm). No manganese, iron, or copper was found, suggesting that any interfering oxidants (assuming they are present) are probably organic, perhaps originating from the local environment. This could not be proven experimentally but is supported by the good correlation between the on-chip analysis and the other methods used.

As shown in Table 2, only six samples had chlorine levels in range (1-4, 6, 12). Three of these were professionally managed (1-3). Therefore, most of the domestic pools were either over or under-dosed with chlorine. This highlights the need for an effective on-line chlorine sensor for domestic pools. Three pools were over-dosed in the range of 4 to 14 ppm (5, 7 and 11). For more than 8 ppm, the S/MO ratio was switched from 3:1 to 1:1 to widen the measurement range. Samples 8, 9, 10, 11c (Table 2) were under-dosed and, in Sample 10, bound chlorine was the major chlorine species (0.4 ppm on-chip). In one instance (Sample 11), the owners had recently purchased the property and were unaware that their chlorinator had failed. The pool was manually dosed prior to purchase (11a) and degraded over two weeks (11b, 11c) to near zero chlorine.

TABLE 2
Swimming pool water measurements

| | | Active Chlorine [ppm] | | | | Total Chlorine [ppm] | | | | pH | | | | |
|---|---|---|---|---|---|---|---|---|---|---|---|---|---|---|
| | | On- | Off- | | | On- | Off- | | | On- | Off- | pH | | |
| ID | Type^{#} | chip | chip | DPD1 | AC7 ® | chip | chip | D2022 | AC7 ® | Chip | chip | electrode | AC7 ® | Notes |
| 1 | P/I | 0.7 | 0.9 | 0.7 | 0.5 | 1.1 | 0.9 | 1.1 | 1 | 6.8 | 6.9 | 7 | 6.8 | — |
| 2 | P/I | 2 | 1.7 | 1.8 | 3 | 2.1 | 1.9 | 2.2 | 3 | 7.4 | 7.5 | 7.6 | 6.8 | — |
| 3 | P/O | 1 | 0.9 | 0.9 | 1 | 1 | 1.1 | 2.7 | 1 | 6.8 | 6.9 | 7 | 6.2 | — |
| 4 | D/O | 1.7 | 1.6 | 1.5 | 1 | 1.5 | 1.5 | 2.8 | 1 | 7.5 | 7.4 | 7.4 | 8.4 | — |
| 5 | D/O | 10 | 10.5 | 10 | 10 | 10.3 | 10 | 10.3 | 10 | 7.3 | 7.5 | 7.4 | 6.8 | High Cl |
| 6 | D/O | 2 | 1.8 | 1.7 | 1 | 1.8 | 1.5 | 1.8 | 1 | 7.4 | 7.4 | 7.4 | 7.2 | — |
| 7 | D/O | 4.8 | 4.6 | 4.6 | 5 | 4.2 | 4.4 | 4.5 | 5 | 7.4 | 7.5 | 7.5 | 7.2 | High Cl |
| 8 | D/O | −0.2 | 0 | −0.1 | 0 | −0.3 | 0 | 0.2 | 0 | 7.5 | 7.6 | 7.6 | 7.2 | Low Cl |
| 9 | D/O | 0.3 | 0 | −0.1 | 0 | 0.2 | 0 | 0 | 0 | 7.5 | 7.5 | 7.6 | 7.2 | Low Cl |
| 10 | D/O | 0.1 | 0 | 0 | 0 | 0.4 | 0.4 | 0.6 | 0 | 7.8 | 7.8 | 7.7 | 7.2 | Low Cl |
| 11a | D/O | 13.6 | 13.5 | 12.8 | 20 | 13.6 | 13.2 | 13 | 10 | 7.2 | 7.2 | 7.2 | 6.8 | High Cl |
| 11b | D/O | 9.7 | 9.5 | 9.2 | 10 | 9.3 | 9.3 | 9.2 | 10 | 7.4 | 7.5 | 7.5 | 7.2 | High Cl |
| 11c | D/O | 0.2 | 0.3 | 0.1 | 0 | 0.3 | 0.2 | 0.1 | 0 | 7.7 | 7.7 | 7.7 | 7.2 | Low Cl |
| 12a | D/O | 3.6 | 3.7 | 3.4 | 3 | 3.8 | 3.5 | 3.9 | 3 | 6.8 | 6.5 | 6.7 | 6.2 | — |
| 12b | D/O | 3 | 2.8 | 2.7 | 3 | 3.1 | 2.8 | 3 | 3 | 6 | 5.9 | 6.1 | 6.2 | — |

Finally, the pH measurements are made. The on-chip method showed good agreement (<0.2 difference) with the precision laboratory pH electrode (FIG. 13). All analysis methods reported that some pools were slightly acidic, compared to the ideal swimming pool pH range (7.2-7.8). The AC7 test strips showed the highest differences compared to the pH electrode, with significant scatter (FIG. 13, triangles). The inaccuracy stems from the visual comparison with the supplied colour chart, which is in steps of 0.4 or 0.6 pH units.

Based on the illustrated design, the sensor will use less than 35 mL of reagent solution over a summer season (3 months, 6 measurements per day). The MO isosbestic point is used to avoid pH effects on the chlorine measurements. Changing the sample/reagent flow ratio permits tuning of the concentration range and sensitivity of the analysis, which is shown to be useful for over-chlorinated pools, e.g. after breakpoint chlorination. For PR, the ratio of absorbance measured at 430 nm and 560 nm is used over the pH range of 6 to 8.5. The on-chip results show excellent agreement with a precision laboratory pH electrode, and outperforms single-use test strips that are often used for domestic pool monitoring, and thereby an accurate and reliable online pool sensor is provided.

Twelve swimming pools were sampled (9 domestic, 2 indoor public, 1 outdoor public) and analysed using the on-chip chlorine and pH methods. The results showed that the on-chip method matches the performance of high-precision analytical methods and out-performs low-cost alternatives. No pre-treatment of the pool samples was necessary in this study. It was found that approximately half of the samples were over or under-chlorinated, posing a health risk to the (domestic) users. In most cases, the owner of the pool was unaware of the imbalanced chemical condition of the pool. In one case, the owner was unaware that their chlorinator was not working at all. These examples highlight the need for a low-cost online microfluidic sensor.

Advantageously, the microfluidic device, equipped with internal flow resistances, does not require re-calibration for pH and is competitive for a precise reading of the chlorine levels in 15 different pool samples.

### REFERENCES

[1] Swimming Pool and Spa Chemistry, https://www.nitt.edu/home/students/facilitiesnservices/sportscenter/swimmingpool/Swim-pool-chemistry.pdf (accessed March 2018)
[2] Public Swimming Pool and Spa Pool Advisory Document, http://www.health.nsw.gov.au/environment/Publications/Swimming-Pool-and-Spa-Advisory-doc.pdf (accessed March 2018)
[3] M. J. Gray, W.-Y. Wholey and U. Jakob, Annu. Rev. Microbiol., 2013, 67, 141-160
[4] S. B. Bharate, S. I. Khan, B. L. Tekwani, M. Jacob, I. A. Khan and I. P. Singh, Bioorg. Med. Chem., 2008, 16, 1328-1336
[5] Swimming pool and spa water chemistry, Missouri Department of Health
[6] White's Handbook of Chlorination, Wiley, 2010
[7] M. V. B. Zanoni, J. J. Sene, H. Selcuk and M. A. Anderson, Environ. Sci. Technol., 2004, 38, 3203-3208.
[8] X. Yao, X. Liu, D. Zhu, C. Zhao and L. Lu, Catal. Commun., 2015, 59, 151-155.
[9] P. Wang, B. Huang, X. Qin, X. Zhang, Y. Dai, J. Wei and M.-H. Whangbo, Angew. Chem. Int. Ed., 2008, 47, 7931-7933.
[10] Irrigation water quality, Department of Primary Industries, https://www.dpi.nsw.gov.au/_data/assets/pdf_file/0005/433643/Irrigation-water-quality.pdf (accessed June 2018)
[11] Water Quality for Crop Production, https://ag.umass.edu/greenhouse-floriculture/greenhouse-best-management-practices-bmp-manual/water-quality-for-crop (accessed June 2018)
[12] L. H. Nowell and J. Hoigné, Water Res., 1992, 26, 599-605.
[13] Y. Feng, D. W. Smith and J. R. Bolton, J. Environ. Eng. Sci., 2007, 6, 277-284.
[14] M. J. Watts and K. G. Linden, Water Res., 2007, 41, 2871-2878
[15] M. J. Gray, W.-Y. Wholey and U. Jakob, Annu. Rev. Microbiol., 2013, 67, 141-160.
[16] K. Murphy, B. Heery, T. Sullivan, D. Zhang, L. Paludetti, K. T. Lau, D. Diamond, E. Costa, N. O'Connor and F. Regan, Talanta, 2015, 132, 520-527.
[17] G. Duffy, I. Maguire, B. Heery, P. Gers, J. Ducrée and F. Regan, Talanta, 2018, 178, 392-399.
[18] Y. Dong, G. Li, N. Zhou, R. Wang, Y. Chi and G. Chen, Anal. Chem., 2012, 84, 8378-8382.
[19] K. Singh and S. K. Mehta, The Analyst, 2016, 141, 2487-2492
[20] Sollo et al., J Am Wat Assoc. vol. 57, pp. 1575 (1965)
[21] in Standard Test Methods of Sampling and Chemical Analysis of Chlorine-Containing Bleaches, ASTM International, 2008, pp. 1-9.
[22] Sollo et al., Environ Sci Tech. vol. 5, pp. 240 (1971)
[23] J.-F. Boily and T. M. Seward, J. Solut. Chem., 2005, 34, 1387-1406
[24] G. Holzner, C. Binder, F. H. Kriel and C. Priest, Langmuir, 2017, 33, 1547-1551
[25] F. H. Kriel, G. Holzner, R. A. Grant, S. Woollam, J. Ralston and C. Priest, Chem. Eng. Sci., 2015, 138, 827-833
[26] B. Jovov, N. K. Wills and S. A. Lewis, Am. J. Physiol.-Cell Physiol., 1991, 261, C1196-C1203
[27] H. Zhang, X. Fan, X. Quan, S. Chen and H. Yu, Environ. Sci. Technol., 2011, 45, 5731-5736
[28] S. Elmas, F. Ambroz, D. Chugh and T. Nann, Langmuir, 2016, 32, 4952-4958
[29] A. Kraft, Platin. Met. Rev., 2008, 52, 177-185
[30] M. E. H. Bergmann and A. S. Koparal, J. Appl. Electrochem., 2005, 35, 1321-1329
[31] M. E. H. Bergmann and J. Rollin, Catal. Today, 2007, 124, 198-203

The reference to any prior art in this specification is not, and should not be taken as, an acknowledgement of any form of suggestion that such prior art forms part of the common general knowledge.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

Please note that the following claims are provisional claims only, and are provided as examples of possible claims and are not intended to limit the scope of what may be claimed in any future patent applications based on the present application. Integers may be added to or omitted from the example claims at a later date so as to further define or re-define the invention.

1. A microfluidic device for measuring pH in a fluid sample, the device comprising:
a sample microfluidic channel disposed on a solid substrate and configured to transfer the fluid sample to be analysed,
a pH indicator microfluidic channel disposed on a solid substrate and configured to transfer a pH indicator solution capable of responding to pH in the fluid sample to produce a pH measurement solution having a response indicative of the pH of the fluid sample,
a mixing microfluidic channel disposed on a solid substrate and in fluid communication with the sample microfluidic channel and the pH indicator microfluidic channel, the mixing microfluidic channel being configured to mix the fluid sample to be analysed with the pH indicator solution under conditions suitable for the pH indicator to respond to pH in the fluid sample to produce a pH measurement solution having a response indicative of the pH, and
an optical reading window in fluid communication with an outlet of the mixing microfluidic channel, through which the response indicative of the pH change can be measured optically.

2. (canceled)

3. The microfluidic device of claim 1, wherein the mixing microfluidic channel is serpentine in form.

4. The microfluidic device of claim 1, wherein the sample microfluidic channel, the pH indicator microfluidic channel and the mixing microfluidic channel are serpentine in form.

5. (canceled)

6. (canceled)

7. The microfluidic device of claim 1, wherein the sample microfluidic channel, the pH indicator microfluidic channel, the mixing microfluidic channel, and if present, the waste microfluidic channel are configured to allow diffusive mixing.

8. The microfluidic device of claim 1, wherein the mixing microfluidic channel is configured so that the residence time from mixing the fluid sample and the pH indicator solution to arriving at the optical reading window is longer than the minimum diffusive mixing time.

9. (canceled)

10. The microfluidic device of claim 1, wherein the microfluidic device comprises a measuring chamber comprising the optical reading window and configured to receive the pH measurement solution and through which the response indicative of pH in the pH measurement solution can be measured optically.

11-15. (canceled)

16. A method of measuring pH in a fluid sample, which includes using the microfluidic device claimed in claim 1.

17. The method of claim 16, wherein the response indicative of the pH is a color change.

18. (canceled)

19. (canceled)

20. The method of claim 17, wherein absorbance measurements from peaks centred at 432 nm, 560 nm, and 650 nm are measured and relative peak intensity is calculated, and the average value used to calculate pH.

21. (canceled)

22. The method of claim 16, wherein the mixing ratio of the fluid sample and the pH indicator solution is 1:1.

23. The method of claim 16, wherein the pH to be measured is in the range between 6 and 8.5.

24-39. (canceled)

40. A microfluidic device for measuring an amount of active chlorine, an amount of total chlorine and pH in a fluid sample, the device comprising:
a sample microfluidic channel disposed on a solid substrate and configured to transfer the fluid sample to be analysed,
a first reagent microfluidic channel disposed on a solid substrate and configured to transfer a first indicator dye solution capable of reacting with any active chlorine in the fluid sample to produce a active chlorine measurement solution having a reduced indicator dye concentration that is indicative of the amount of active chlorine in the fluid sample,
a second reagent microfluidic channel disposed on a solid substrate and configured to transfer a second indicator dye solution capable of reacting with any total chlorine in the fluid sample to produce a total chlorine measurement solution having a reduced indicator dye concentration that is indicative of the amount of total chlorine in the fluid sample,
a third reagent microfluidic channel disposed on a solid substrate and configured to transfer a pH indicator solution capable of responding to pH in the fluid sample to produce a pH measurement solution having a response indicative of the pH,
a mixing microfluidic channel disposed on a solid substrate and in fluid communication respectively with the sample microfluidic channel, the first reagent microfluidic channel, the second reagent microfluidic channel and the third reagent microfluidic channel, which is configured to mix the fluid sample separately with the first indicator dye solution, the second indicator dye solution and the pH indicator solution under conditions suitable for some of the indicator dye in the first indicator dye solution to react with any active chlorine in the fluid sample to produce an active chlorine measurement solution having a reduced indicator dye concentration that is indicative of the amount of active chlorine in the fluid sample, suitable for some of the indicator dye in the second indicator dye solution to react with any total chlorine in the fluid sample to produce a total chlorine measurement solution having a reduced indicator dye concentration that is indicative of the amount of total chlorine in the fluid sample, and suitable for the pH indicator to respond to pH in the fluid sample to produce a pH measurement solution having a response indicative of the pH, and
an optical reading window located downstream of the mixing microfluidic channel, through which the amount of active chlorine, the amount of total chlorine and pH in the fluid sample can be optically measured.

41-43. (canceled)

44. The microfluidic device of claim 40, wherein the microfluidic device is a multilayer microfluidic device comprising first and second outer chips and first and second intermediate chips and wherein the sample microfluidic channel, the first reagent microfluidic channel, the second reagent microfluidic channel, and the third reagent microfluidic channel are disposed on the first intermediate chip, and the mixing microfluidic channel and the optical reading window are disposed on the second intermediate chip.

45. (canceled)

46. The microfluidic device of claim 40, wherein the flow resistance of the sample microfluidic channel, the first reagent microfluidic channel, the second reagent microfluidic channel, the third reagent microfluidic channel, and the mixing microfluidic channel are sufficient to minimize backflow of the fluid sample, the first indicator dye solution, the second indicator dye solution and the pH indicator solution during operation.

47. The microfluidic device of claim 40, wherein the sample microfluidic channel, the first reagent microfluidic channel, the second reagent microfluidic channel, and the third reagent microfluidic channel, and the mixing microfluidic channel are serpentine in form.

48. (canceled)

49. The microfluidic device of claim 40, wherein the cross-section of the mixing microfluidic channel is of greater size than those of the sample microfluidic channel, the first reagent microfluidic channel, the second reagent microfluidic channel, the third reagent microfluidic channel and, if present, the waste microfluidic channel.

50. (canceled)

51. The microfluidic device of claim 40, wherein the mixing microfluidic channel is configured so that the residence time from mixing the fluid sample separately with the first indicator dye solution, the second indicator dye solution and the pH indicator solution to arriving at the optical reading window is longer than the characteristic time for diffusive mixing.

52-29. (canceled)

60. A method of measuring an amount of active chlorine, an amount of total chlorine and pH in a fluid sample, which includes using the microfluidic device claimed in claim 40.

61-79. (canceled)

80. The method of claim 60, wherein the detection wavelength for measuring an amount of active chlorine and/or an amount of total chlorine is set at the isosbestic point of methyl orange at 469 nm.

81. The method of claim 80, wherein the absorbance at 650 nm as background was subtracted from the absorbance at 469 nm, and the average values are used for measuring an amount of active chlorine and an amount of total chlorine.

82. (canceled)
